(19) United States
(12) Patent Application Publication
(10) Pub. No.: US 2026/0193588 A1
(43) Pub. Date: Jul. 9, 2026

(54) AUTOMATED CELL ANALYSER AND METHOD

(71) Applicant: PROVECTUS IP PTY LTD, Noosaville (AU)

(72) Inventors: Nusqe SPANTON, Noosa (AU); Preston TOOLE, Sunshine Beach (AU); Andreas HUEMER, Coorparoo (AU); Mikolaj WRZESINSKI, Noosaville (AU); Benjamin SZABO-VIRAG, Sunshine Coast (AU); Lucas JURVILLIER, Caloundra West (AU); Owen BAWDEN, Canberra (AU); Mahyar OSANLOUY, Sydney (AU); Lisa HUTCHINS, Tewantin (AU); Mateusz STANKIEWICZ, Poznan (PL); Michal ANTOSZKIEWICZ, Borkowo (PL)

(21) Appl. No.: 19/134,040

(22) PCT Filed: Oct. 20, 2023
(86) PCT No.: PCT/AU2023/051052

§ 371 Date: May 29, 2025

(30) Foreign Application Priority Data
Dec. 1, 2022 (AU) 2022279500

(51) Int. Cl. C12M 1/36; C12M 1/26; G06V 10/14; G06V 10/44; G06V 10/70; G06V 20/69

(52) U.S. Cl. CPC C12M 41/48; C12M 33/00; G06V 10/14; G06V 10/44; G06V 10/70; G06V 20/693; G06V 20/695

(57) ABSTRACT

An automated cell analyser and method for determining a property of a cell, preferably a microalgal cell. In an embodiment, the method includes the steps of: (1) adding a cell sample to a microfluidic device; (2) capturing one or more images of at least one cell of the sample located within the microfluidic device; (3) carrying out image recognition of the one or more images to thereby determine a property of the at least one cell; and (4) cleaning the microfluidic device prior to adding a new cell sample to the microfluidic device, wherein: steps (3) and (4) need not be carried out in the stated order; steps (1) to (4) are preferably repeated using new cell samples; and at least steps (2) to (4) are automated.

### RELATED APPLICATIONS

This application claims priority of Australian Patent Application No. 2022279500, filed 1 Dec. 2022, the entire contents of which are incorporated herein by reference.

### FIELD OF THE INVENTION

This invention broadly relates to an automated cell analyser and method for determining a property of a cell. The invention is primarily described with reference to analysing microalgae, but this description is used by way of example only and the invention is not limited to this example.

### BACKGROUND

Microalgae is typically grown in bioreactors. As part of this process, it is usually necessary to monitor growth of the microalgae as well as other properties of the cultured microalgae, such as cell count and cell health.

The standard conventional monitoring method involves a technician manually withdrawing a sample of microalgae culture from the bioreactor and visualising microalgae cells under a microscope. The technician then manually determines the required property of the cultured microalgae, such as cell count or cell health.

This standard conventional monitoring method, which is also used for other cell types in culture, is both extremely time consuming and may lack accuracy or precision due to human error.

### OBJECT OF THE INVENTION

It would be advantageous to provide an automated cell analyser or method for determining a property of a cell that minimises or overcomes a disadvantage of the standard conventional monitoring method mentioned above, or to provide consumers with a useful choice.

### SUMMARY OF THE INVENTION

According to a first aspect of the present invention, there is provided an automated cell analyser for determining a property of a cell, comprising:
a sampling system comprising a microfluidic device for receiving a sample comprising at least one cell;
an imaging system associated with the microfluidic device, adapted to capture one or more images of the at least one cell of the sample located within the microfluidic device;
an image recognition system for recognising the at least one cell within the one or more captured images of the sample, to thereby determine a property of the at least one cell; and
a controller for automating at least the imaging and image recognition systems.

The at least one cell can be of any suitable type. Preferably, the sample comprises a plurality of cells and one or more properties of one or more of those cells is determined. In some embodiments, suitable cell types include those of unicellular organisms (single-cell organisms), protozoans, unicellular prokaryotes, unicellular eukaryotes and of the Kingdom Protista. Such cells can be those of bacteria, protozoa, fungi, algae or archaea. In some embodiments, suitable cell types include those of microalgae. In some embodiments, suitable cell types include those of multi-cellular organisms when grown in a unicellular manner or can be separated into separate cells, such as when grown in culture. Such cells include plant and animal cells, such as mammalian and insect cells.

Any suitable type of sample can be used. Typically, the sample will be taken of a cell culture in which the at least one cell is suspended in a suspension, growth or culture medium. Examples include freshwater and saltwater mediums. Suitable suspension, growth and culture mediums for prokaryotic and eukaryotic cells are found in handbooks such as the ‘Cell Culture Basics Handbook’ by ThermoFisher Scientific/Gibco (https://assets.thermofisher.com/TFS-Assets/BID/Handbooks/gibco-cell-culture-basics-handbook.pdf), the entire contents of which are incorporated herein by reference.

Suitable suspension, growth and culture mediums for microalgae/algae are found in Tan et al. (Tan J S, Lee S Y, Chew K W, Lam M K, Lim J W, Ho S H, Show P L. A review on microalgae cultivation and harvesting, and their biomass extraction processing using ionic liquids. Bioengineered. 2020 December; 11(1):116-129. doi: 10.1080/21655979.2020.1711626. PMID: 31909681; PMCID: PMC6999644), and, de Carvalho et al. (Julio Cesar de Carvalho, Eduardo Bittencourt Sydney, Lorenzo Ferrari Assú Tessari, Carlos Ricardo Soccol, Chapter 2-Culture media for mass production of microalgae, Editor(s): Ashok Pandey, Jo-Shu Chang, Carlos Ricardo Soccol, Duu-Jong Lee, Yusuf Chisti, In Biomass, Biofuels, Biochemicals, Biofuels from Algae (Second Edition), Elsevier, 2019, Pages 33-50, ISBN 9780444641922, https://doi.org/10.1016/B978-0-444-64192-2.00002-0), the entire contents of which are incorporated herein by reference.

Any suitable type of microalgae can be used, including from the following genera: Cyanophyta, Prochlorophyta, Rhodophyta, Chlorophyta, Heterokontophyta, Tribophyta, Glaucophyta, Chlorarachniophytes, Euglenophyta, Euglenoids, Haptophyta, Chrysophyta, Cryptophyta, Cryptomonads, Dinophyta, Dinoflagellata, Pyrmnesiophyta, Bacillariophyta, Xanthophyta, Eustigmatophyta, Raphidophyta, Phaeophyta, and Phytoplankton. A microalgae may also be a microalgae species including, but not limited to, Chlamydomonas reinhardtii, Dunaliella salina, Nannochloropsis salina, Nannochloropsis occulata, Scenedesmis dimorphus, Scenedesmus obliquus, Dunaliella tertiolecta, or Haematococcus pluvialis.

Additional non-limiting examples of microalgae species that can be used include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tennis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

Examples of suitable species include, but are not limited to: Chaetoceros calcitrans; Chaetoceros gracilis; Nitzchia Closterium; Phaeodactylum tricornutum; Thalassiosira pseudonana; Cylindrogheca fusiformis; Dunaliella tertiolecta; Tetraselmis suecica; Chlorella vulgaris; Dunaliella salina; Nannochloropsis oculatal; Isochrysis galbana; Pavlova lutheri; Pavlova salina; Cryptomonas rufescens; Nostoc commune; Spirulina platensis; Arthrospira platentsis; Aphanizomenon flos-aquae; and, Euglena gracilis.

Preferred examples include Chlamydomonas reinhardtii, Porphyridium cruentum and Chlorella vulgaris.

In some embodiments, the sample contains at least one prokaryotic or eukaryotic cell. In some embodiments, the sample contains at least one microalgae cell. In some embodiments, the sample is of a cell culture.

The sampling system can be of any suitable size, shape and construction.

In some embodiments, the sampling system is for withdrawing and conveying a sample of a cell culture. In some embodiments, the sampling system is for withdrawing and conveying a sample of a cell culture, said sampling system comprising the microfluidic device, and a sampling line for conveying the sample between a container adapted to contain the cell culture and the microfluidic device.

In some embodiments the sampling system comprises the container adapted to contain the cell culture (‘cell culture container’).

In some embodiments, the sampling line comprises an inlet connected to an outlet of the cell culture container and an outlet connected to the microfluidic device.

The cell culture container can be of any suitable size, shape and construction. In some embodiments the container is a microtitre well or plate, test tube, flask, bottle, tank, vessel, pond, bioreactor or photobioreactor—basically anything that can hold a cell culture. In some embodiments the container has a volume of about 200 L.

The microfluidic device can be of any suitable size, shape and construction. In some embodiments, the microfluidic device is in the form of a chip comprising a chip body and at least one channel extending along and/or within the chip body. A suitable chip having acceptable optical quality is sold, for example, by Microfluidic ChipShop at https://www.microfluidic-chipshop.com/. The channel can have an inlet and an outlet. In some embodiments, the channel has a depth of about 20-100 microns, preferably about 30-50 microns, more preferably about 30 microns. The channel depth will depend on the size/diameter of the cells, typically being in the order of approximately 1 to 30 microns, more preferably 1 to 15 microns. The channel inlet can be connected to the outlet of the sampling line.

In some embodiments, the sampling system can comprise more than one microfluidic device (ie. 2, 3, 4, 5, 6, 7, 8, 9, 10 or more), each being adapted to receive a separate sample. Each channel of a microfluidic device can have an inlet and outlet.

In some embodiments, the sampling system can comprise more than one sampling line. Each sampling line can be connected to a separate microfluidic device. Each sampling line can be connected to a separate cell culture container.

Any suitable sample size can be used. Typically, the sample size is in the microlitre to millilitre range. Typically, the channel of the microfluidic device accommodates a sample size in the microlitre range, preferably between about 2.5 to 5.0 microlitres.

The sampling line can be of any suitable size, shape and construction. In some embodiments, the sampling line can comprise one or more lengths of pipe, tubing and/or hose connected together. In some embodiments, the sampling line can comprise a manifold. In some embodiments, the sampling line can have an internal diameter of approximately 0.6 mm. In some embodiments, the sampling line can comprise silicone tubing, preferably platinum cured silicone.

The sampling system can comprise one or more electric valves, taps and/or flow diverters for controlling or regulating the flow of sample/liquid between the culture container and the microfluidic device, as well as from the microfluidic device to a waste container. The sampling system can comprise one or more connectors, fittings etc. for connecting components of the system together.

The sampling system can comprise at least one pump for conveying the sample to the microfluidic device. The sampling system can comprise at least one pump for withdrawing the sample and conveying the sample to the microfluidic device. Any suitable type of pump can be used. In some embodiments, at least one pump is located in-line downstream of the microfluidic device, such that it can draw the sample from the microfluidic device and for some embodiments also from the cell culture container. In some embodiments the pump can further convey the sample from the microfluidic device to the waste container. In some embodiments the pump can convey the sample from the waste container to the microfluidic device and for some embodiments further to the cell culture container. Preferably the pump is a peristaltic pump, providing a sample flow rate of approximately 2 to 17 mL/min.

The sampling system can comprise a pump line having a first end connected to the pump and a second end connected to the channel outlet. The pump line can be of any suitable size, shape and construction. In some embodiments, the pump line can comprise one or more lengths of pipe, tubing and/or hose connected together. In some embodiments, the pump line can comprise a manifold. In some embodiments, the pump line can have an internal diameter of approximately 0.6-1 mm. In some embodiments, the pump line can comprise silicone tubing, preferably platinum cured silicone.

The sampling system can comprise a waste container and a waste line having a first end connected to the pump and a second end connected to the waste container. The waste container can collect part or all of the sample, as required.

The waste container can be of any suitable size, shape and construction. In some embodiments the waste container is a test tube, flask, bottle, tank, or vessel-basically anything that can hold liquid waste.

The waste line can be of any suitable size, shape and construction. In some embodiments, the waste line can comprise one or more lengths of pipe, tubing and/or hose connected together. In some embodiments, the waste line can comprise a manifold. In some embodiments, the waste line can have an internal diameter of approximately 1 mm. In some embodiments, the waste line can comprise silicone tubing, preferably platinum cured silicone.

The sampling system can convey the sample to the microfluidic device such that cells of the sample are located within the channel during image capture. Although the sample can flow continuously through the channel during image capture, preferably, sample flow is interrupted and stopped at the time an image is to be captured.

In some embodiments, the sampling system comprises a pipetting robot for adding a sample to the microfluidic device (or samples to microfluidic devices).

In some embodiments, the sampling system comprises a pipetting robot for withdrawing a sample of a cell culture and conveying that sample to the microfluidic device (or samples of difference cell cultures to different microfluidic devices). The pipetting robot can be of any suitable construction, such as those sold by Opentrons (opentrons.com), Bio Molecular Systems (biomolecularsystems.com) and Integra Biosciences (integra-biosciences.com).

In some embodiments, the sampling system comprises a user/technician manually adding a sample to the microfluidic device. This can be achieved in any suitable way, typically using a pipetting step.

In some embodiments, the sampling system comprises a user/technician manually withdrawing a sample of a cell culture and conveying that sample to the microfluidic device. This can be achieved in any suitable way, typically using a pipetting step.

The imaging system can be of any suitable size, shape and construction. In some embodiments the imaging system comprises an imaging device. The imaging device can comprise both optics and electronics. Preferably the imaging device comprises a microscope, having its optics in close proximity of the channel of the microfluidic device, such that cells within the channel can be imaged. In this embodiment, the term ‘microfluidic device associated with an imaging system’, means that the microfluidic device channel is in close proximity of the optics of the imaging device/microscope such that any cells within the channel can be imaged. Preferably the imaging device comprises an inverted light microscope.

The imaging device can comprise a light source for illuminating the cells within the channel. Any suitable light source can be used. The light source can comprise a light such as an LED. The light source can comprise a light housing. The light source can comprise a positioning mechanism for adjusting the distance/position of the light relative to the channel, such that a focal point of the light can be adjusted. The positioning mechanism can be manually actuated or motorised/electronic for automatic adjustment. For example, the positioning mechanism can comprise a rack and pinion mechanism connected to the light housing for moving the light substantially vertically relative to the channel.

Preferably, the imaging device comprises optics that can be adjusted for magnification, focus, contrast and/or resolution, preferably by way of motors, actuators, solenoids and the like. Preferably, the optics comprise a lens located within a lens housing. The optics can comprise a positioning mechanism for adjusting the distance of the lens relative to the channel, such that a focal length of the lens can be adjusted. The positioning mechanism can be automatically adjusted. The positioning mechanism can be motorised/electronic for automatic adjustment. For example, the positioning mechanism can comprise a stepper motor coupled with the lens via an endless belt (for infinite positioning), for moving the lens substantially vertically relative to the channel so that the lens can focus on cells located at different depths within the channel. Focussing of the lens can be carried out automatically. This can be carried out in any suitable way. For example, an API server and web interface for the OpenFlexure Microscope for autofocussing is found at the URL https://openflexure.org/projects/microscope/install (Raspbian-OpenFlexure-full graphical microscope control software). Python code for autofocussing the microscope is found at https://gitlab.com/openflexure/openflexure-microscope-server/-/blob/master/openflexure_microscope/api/default_extensions/autofocus.py (autofocus.py).

In some embodiments, an inverted microscope is used whereby the light source is located above the channel and the optics/lens is located beneath the channel.

In some embodiments, the imaging system or imaging device can comprise a digital camera for capturing still images and/or video recordings of the sample. The camera can be of any suitable, size, shape and construction. The camera can comprise optics and electronics.

In some embodiments, the camera is connected to the imaging device's optics such that images of cells can be captured whilst located within the channel. For example, the camera can be connected to or housed within the lens housing below the lens.

The imaging system can communicate the captured images to the image recognition system in any suitable way. In some embodiments, the imaging system can communicate captured image data either through a wired connection or wirelessly (e.g., Wi-Fi (WLAN) communication, Satellite communication, RF communication, infrared communication, or Bluetooth™), using a wireless transceiver, with a standalone computer, a computer network, a website interface, smart phone or other electronic device (ie. a receiver), for example.

In some embodiments, a single image of the sample is taken. In some embodiments, multiple images of the sample are taken, such as up to 5 images. In some embodiments, a video is taken. Depending on the property of the cell to be determined, a single captured image or an average of multiple captured images (or video image stills) can be used for image recognition.

The image recognition system can be of any suitable construction. In some embodiments, the image recognition system comprises image recognition software that, when executed, processes captured image data of the cells to determine a property of the cell. The image recognition system can utilise artificial intelligence (AI)/machine learning to determine a property of the cell.

In some embodiments the imaging system and image recognition system can be in the form of a computational microscope for capturing still images and/or video recordings of the sample within the channel and for carrying out image recognition.

In some embodiments captured image data is communicated to a receiver and image recognition software is executed on the receiver. The receiver can have a CPU. The receiver can have memory. The receiver can have a display screen. The receiver can have a user-friendly interface. The receiver can have a printing function. The receiver can have a data logging or other data recording function. The receiver can be a standalone computer, a computer network, a website interface, smart phone or other electronic device, for example.

In some embodiments, the image recognition system carries out a first cell detection step based on its ability to detect cell contour. This step can be carried out in any suitable way, including as described in the thesis of J. Ajala, the entire contents of which are incorporated herein by reference (Ajala, John, “Object Detection and Recognition Using YOLO: Detect and Recognize URL(s) in an Image Scene” (2021). Culminating Projects in Computer Science and Information Technology. 37. https://repository.stcloudstate.edu/csit_etds/37). In some embodiments, the one or more captured images of the sample in the first cell detection step are analysed to detect objects and/or small particles. In some embodiments, the image recognition system discriminates microalgae cells from other small particles, such as debris, small stains and microbubbles. In some embodiments, the one or more captured images of the sample are split into tile sections and/or scaled to a predetermined size before the first cell detection step. In some embodiments, the first cell detection step is carried out by a convolutional neural network (CNN). In some embodiments, the CNN has a 24-layer architecture with two fully connected layers. In some embodiments, CNN predicts multiple bounding boxes for each potential cell identification in the one or more captured images. In some embodiments, the predictions for the bounding boxes are provided a confidence value. In some embodiments the CNN uses non-maximum suppression (NMS) to remove overlapping bounding boxes with higher confidence values. In some embodiments, the image recognition system carries out a subsequent cell property recognition step. In some embodiments, the cell property recognition step comprises a counting step, to count the number of cells present in the image. In some embodiments, the image recognition system excludes objects and/or small particles other than microalgae from being analysed during the cell property recognition step. In some embodiments, the image recognition system generates images of one or more microalgae cells detected at the first cell detection step, and these images are analysed at subsequent cell property recognition step. In some embodiments, the cell property recognition step comprises a cell-feature recognition step whereby: (a) parent and smaller daughter cells in close proximity of each other are recognised; (b) cell surface area and/or cell volume is calculated; (c) cell clumps are recognised; (d) cell colour is recognised; (e) two different cell types (eg. algal and bacterial) are discriminated between; or, (f) the cell species or stain type is identified. Steps (a) to (f) can be carried out in any suitable way, including as described in the following publications: CN114170597; CN114511851; CN114511530; CN114495098; CN114418995; CN114418994; CN114067114; CN114004823; CN113987958; CN113537405; CN113128385; CN113092346; WO2021115259; WO2021115239; CN112784748; CN111583178; CN111007221; KR20200020412; CN110532941; CN110487705; KR20190114241; KR20190075696, CN110287990; CN110245562; ES2685220; CN108256533; CN106841072; CN104331712; FR3002355; WO02099734; CN113533171; Ajala, John, “Object Detection and Recognition Using YOLO: Detect and Recognize URL(s) in an Image Scene” (2021). Culminating Projects in Computer Science and Information Technology. 37. https://repository.stcloudstate.edu/csit_etds/37; Ronneberger O, Fischer P, Brox T (2015). “U-Net: Convolutional Networks for Biomedical Image Segmentation”. arXiv:1505.04597. In some embodiments, the cell property recognition step is carried out by a CNN.

The cell detection step can employ artificial intelligence (AI)/machine learning. Preferably the AI determines whether a cell is present or not by detecting at least one cell contour. Preferably, the AI is trained to recognise cell contours by being provided with a set of reference images showing positive and negative examples with any visible cell contours and errors marked.

The property recognition step can employ artificial intelligence (AI)/machine learning. Preferably, the AI is trained to recognise cell properties by being provided with a set of reference images showing positive and negative examples of parent and smaller daughter cells in close proximity of each other, cell surface area and/or cell volume, cell clumps, cell colour, cell types, cell species and/or strain types.

Preferably, a user/technician can request the cell property (or properties) that needs to be determined.

In some embodiments, the image recognition system controls focussing of the imaging device's lens such that the lens automatically scans the depth of the channel and preferentially focuses on cells within the channel. This focussing and scanning step can employ artificial intelligence (AI)/machine learning. In some embodiments, the image recognition system causes the imaging device lens to focus on the largest number of cells within the channel. This focussing step can employ artificial intelligence (AI)/machine learning. In some embodiments, the image recognition system causes the lens to focus through different depths/planes of the channel, until it locates the highest number of cells. This focussing step can employ artificial intelligence (AI)/machine learning.

In some embodiments, the image recognition system causes the lens to focus through different depths/planes of the channel, until it locates the highest number of cells, and then uses that same depth/plane for image capture of other subsequent samples added to the channel (or samples in the channels of other microfluidic devices).

In embodiments where more than one microfluidic device is used, the imaging system is capable of movement relevant to the microfluidic devices such that images of cells within all devices can be captured.

The automated cell analyser can comprise a pre-sampling wetting system for wetting the microfluidic device prior to sampling, and in some embodiments also prior to conveying of the sample. In some embodiments the pre-sampling wetting system wets the microfluidic device prior to sampling and conveying of the sample. In some embodiments the pre-sampling wetting system wets the microfluidic device as well as the sampling line or part thereof, prior to sampling and conveying of the sample.

The pre-sampling wetting system can be of any suitable size, shape and construction. In some embodiments, the pre-sampling wetting system comprises a container adapted to contain a wetting agent, and a wetting line for conveying the wetting agent from the wetting agent container to the sampling line and/or microfluidic device.

The wetting agent container can be of any suitable size, shape and construction. In some embodiments the wetting agent container is a microtitre well or plate, test tube, flask, bottle, tank, or vessel—basically anything that can hold a wetting agent.

Any suitable type of wetting agent can be used. In some embodiments, the wetting agent is water or other polar solvent, such as ethyl alcohol.

The wetting line can have an inlet connected to an outlet of the wetting agent container, and an outlet connected to the sampling line upstream of the microfluidic device or to the microfluidic device itself.

The wetting line can be of any suitable size, shape and construction. In some embodiments, the wetting line can comprise one or more lengths of pipe, tubing and/or hose connected together. In some embodiments, the wetting line can comprise a manifold. In some embodiments, the wetting line can have an internal diameter of approximately 0.6 mm. In some embodiments, the wetting line can comprise silicone tubing, preferably platinum cured silicone.

The pre-sampling wetting system can comprise one or more electric valves, taps and/or flow diverters for controlling or regulating the flow of wetting agent/liquid between the wetting agent container and the microfluidic device, as well as from the microfluidic device to a waste container. The pre-sampling wetting system can comprise one or more connectors, fittings etc. for connecting components of the system together.

The pre-sampling wetting system can comprise at least one pump for conveying the wetting agent to the microfluidic device, optionally via the sampling line. Any suitable type of pump can be used. In some embodiments, at least one pump is located in-line downstream of the microfluidic device, such that it can draw the wetting agent from the wetting agent container to the microfluidic device. Preferably the pump is a peristaltic pump, providing a sample flow rate of approximately 2 to 17 mL/min.

The pre-sampling wetting system can comprise a pump line having a first end connected to the pump and a second end connected to the channel outlet.

The pump line can be of any suitable size, shape and construction. In some embodiments, the pump line can comprise one or more lengths of pipe, tubing and/or hose connected together. In some embodiments, the pump line can comprise a manifold. In some embodiments, the pump line can have an internal diameter of approximately 0.6-1 mm. In some embodiments, the pump line can comprise silicone tubing, preferably platinum cured silicone.

The pre-sampling wetting system can comprise a waste container and a waste line having a first end connected to the pump and a second end connected to the waste container. The waste container can collect part or all of the wetting agent, if necessary.

The waste container can be of any suitable size, shape and construction. In some embodiments the waste container is a bottle, tank or vessel-basically anything that can hold liquid waste.

The waste line can be of any suitable size, shape and construction. In some embodiments, the waste line can comprise one or more lengths of pipe, tubing and/or hose connected together. In some embodiments, the waste line can comprise a manifold. In some embodiments, the waste line can have an internal diameter of approximately 1 mm. In some embodiments, the waste line can comprise silicone tubing, preferably platinum cured silicone.

In some embodiments where more than one microfluidic device is used, the pre-sampling wetting system can comprise more than one wetting line, each wetting line being connected to a different microfluidic device, or for some embodiments connected to different sampling lines.

In some embodiments, the automated cell analyser can comprise a sample-cleaning system for cleaning the microfluidic device and for some embodiments also the sampling line or part thereof, after the imaging step.

In some embodiments, the sample-cleaning system can remove the sample or most of the sample from the microfluidic device and for some embodiments also from the sampling line after the imaging step.

In some embodiments, the sample-cleaning system can flush the microfluidic device and for some embodiments also the sampling line or part thereof with at least one type of fluid. The fluid can be a gas and/or a liquid.

In some embodiments, the sample-cleaning system can flush the microfluidic device and for some embodiments also the sampling line with a gas such as air. In some embodiments, an air pump can be used. Preferably, a pump located downstream of the microfluidic device can displace the sample from the microfluidic device and for some embodiments also from the sampling line using air, and for some embodiments return the sample or most of the sample to the cell culture container.

In some embodiments, the sample-cleaning system can flush the microfluidic device and for some embodiments also at least part of the sampling line with a cleaning agent.

Preferably, the sample-cleaning system can both flush the microfluidic device and for some embodiments also the sampling line with a gas such as air, and further flush the microfluidic device and for some embodiments also at least part of the sampling line with a cleaning agent.

The sample-cleaning system can be of any suitable size, shape and construction. In some embodiments, the sample-cleaning system comprises a container adapted to contain a cleaning agent, and a cleaning line for conveying the cleaning agent from the cleaning agent container to the microfluidic device and for some embodiments also to the sampling line.

Any suitable type of cleaning agent can be used. In some embodiments, the cleaning agent is water or other polar solvent, such as ethyl alcohol.

The cleaning agent container can be of any suitable size, shape and construction. In some embodiments the cleaning agent container is a microtitre well or plate, test tube, flask, bottle, tank, or vessel—basically anything that can hold a cleaning agent.

In some embodiments, both the wetting and cleaning agents are water.

The cleaning line can have an inlet connected to an outlet of the cleaning agent container, and an outlet connected to the sampling line upstream of the microfluidic device, or to the microfluidic device itself.

The cleaning line can be of any suitable size, shape and construction. In some embodiments, the cleaning line can comprise one or more lengths of pipe, tubing and/or hose connected together. In some embodiments, the cleaning line can comprise a manifold. In some embodiments, the cleaning line can have an internal diameter of approximately 0.6 mm. In some embodiments, the cleaning line can comprise silicone tubing, preferably platinum cured silicone.

The sample-cleaning system can comprise one or more electric valves, taps and/or flow diverters for controlling or regulating the flow of cleaning agent/liquid between the cleaning agent container and the microfluidic device, as well as from the microfluidic device to a waste container. The sample-cleaning system can comprise one or more connectors, fittings etc. for connecting components of the system together.

The sample-cleaning system can comprise at least one pump for conveying the cleaning agent to the microfluidic device and for some embodiments also via the sampling line or part thereof. Any suitable type of pump can be used. In some embodiments, at least one pump is located in-line downstream of the microfluidic device, such that it can draw the cleaning agent from the cleaning agent container to the microfluidic device. Preferably the pump is a peristaltic pump, providing a sample flow rate of approximately 2 to 17 mL/min.

The sample-cleaning system can comprise a pump line having a first end connected to the pump and a second end connected to the channel outlet.

The pump line can be of any suitable size, shape and construction. In some embodiments, the pump line can comprise one or more lengths of pipe, tubing and/or hose connected together. In some embodiments, the pump line can comprise a manifold. In some embodiments, the pump line can have an internal diameter of approximately 0.6-1 mm. In some embodiments, the pump line can comprise silicone tubing, preferably platinum cured silicone.

The sample-cleaning system can comprise a waste container and a waste line having a first end connected to the pump and a second end connected to the waste container. The waste container can collect part or all of the cleaning agent, if necessary.

The waste container can be of any suitable size, shape and construction. In some embodiments the waste container is a bottle, tank or vessel-basically anything that can hold liquid waste.

The waste line can be of any suitable size, shape and construction. In some embodiments, the waste line can comprise one or more lengths of pipe, tubing and/or hose connected together. In some embodiments, the waste line can comprise a manifold. In some embodiments, the waste line can have an internal diameter of approximately 1 mm. In some embodiments, the waste line can comprise silicone tubing, preferably platinum cured silicone.

In some embodiments, where more than one microfluidic device is used, the sample-cleaning system can comprise more than one cleaning line, each cleaning line being connected to a different microfluidic device, or for some embodiments connected to different sampling lines. The controller for automating the systems can be of any suitable construction.

The controller can comprise a controller housing. The controller housing can be of any suitable size, shape and construction, and can be made of any suitable material or materials.

The controller can comprise logic circuitry such as a PLC, microprocessor or microcontroller. The logic circuitry can be contained within the controller housing. The controller can be configured logic in the form of reprogrammable software or hardcoded software executed by a microcontroller. Alternatively, the controller can be configured with hardcoded logic in the form of an application specific integrated circuit, or programmable logic in the form of a field programmable gate array. Hardcoded logic can be incorporated in conjunction with a microcontroller or in place of a microcontroller.

The controller can be reprogrammable by a user, or by a connected controller, and be suitably configured for any design and operating conditions.

The controller can utilise artificial intelligence (AI)/machine learning to carry out automation. That is, one or more tasks/functions carried out by the controller can utilise artificial intelligence (AI)/machine learning. In one embodiment, conditions can be set to guide the AI/machine learning.

The controller can comprise contacts or electrical sockets for the leads/contacts of the pump/s, valve/s flow diverter/s, actuator/s, motor/s, imaging device's electricals/electronics, and image recognition system.

Preferably, the controller can automate the imaging, image recognition, and sample-cleaning systems. More preferably, the controller can automate the imaging, image recognition, sample-cleaning, and sampling systems. Even more preferably, the controller can automate the imaging, image recognition, sample-cleaning, sampling, and pre-sampling wetting systems.

The controller can be connectable to a power supply.

The controller can be electrically connected to and control electrical/electronic components of the automated cell analyser, such as the pump/s, valve/s, flow diverter/s, tap/s, solenoid/s, actuator/s, motor/s, imaging device, camera and/or image recognition system.

The controller can comprise a microcontroller electrically connected to the pump/s, valve/s flow diverter/s, actuator/s, motor/s, and imaging device's electricals/electronics etc, for control thereof.

The controller can comprise a CAN/LIN communication interface or bus, enabling communication between the microcontroller and other applications, devices or user interface.

The controller can comprise controller software.

The controller can comprise a user interface for setting parameters and to allow real time/live time viewing of operations.

The controller or logic circuit can communicate either through a wired connection or wirelessly (e.g., Wi-Fi (WLAN) communication, Satellite communication, RF communication, infrared communication, or Bluetooth™) via a wireless transceiver, with a standalone computer, a computer network, a website interface, smart phone or other electronic device (ie. a receiver), for example.

The controller can have a data logging or other data recording function, or communicate with a receiver having a data logging or other data recording function. The receiver can have a CPU. The receiver can have memory. The receiver can have a display screen. The receiver can have a user-friendly interface. The receiver can have a printing function.

Any suitable cell property can be determined. For example, the property can be one or more of: the degree of cell growth; cell count; cell health; cell morphology; cell/organism species identification; cell/organism strain identification; the level of recombinant protein expression by the cell; the level of biomolecule (eg. amino acid, peptide, protein, carbohydrate, sugar, lipid, oil, nucleic acid) expressed by the cell; cell maturity; and, one or more traits of the cell.

According to a second aspect of the present invention, there is provided an automated method of determining a cell property, comprising:
at least one sampling step, comprising conveying a sample containing at least one cell to a microfluidic device associated with an imaging system;
at least one imaging step, comprising using the imaging system to capture one or more images of the at least one cell of the sample located within the microfluidic device; and
at least one image recognition step, comprising recognising the at least one cell within the one or more captured images of the sample, to thereby determine a property of the cell, wherein at least the imaging and image recognition steps are automated.

In some, embodiments, the sampling step is also automated.

In some embodiments, the sampling step, imaging step, image recognition step and any other steps described herein can be carried out any number of times, for determining the cell properties of further samples. For example, cell cultures can be periodically sampled for adequate growth.

The method of the second aspect can be carried out using the automated cell analyser according to the first aspect, in which case, according to a third aspect of the present invention, there is provided an automated method of determining a property of a cell, comprising:
at least one sampling step, comprising conveying a sample containing at least one cell to a microfluidic device associated with an imaging system;
at least one imaging step, comprising using the imaging system to capture one or more images of cells of the sample located within the microfluidic device; and
at least one image recognition step, comprising recognising the at least one cell within the one or more captured images of the sample, to thereby determine a property of the cell, wherein:
the sampling step is carried out using a sampling system;
the imaging step is carried out using an imaging system;
the image recognition step is carried out using an image recognition system; and,
the imaging and image recognition steps are automated using a controller.

Hence, features of the first aspect equally apply to the second and third aspects. Features of the first aspect may be rewritten as method features of the second and third aspects, and vice-versa.

The at least one sampling step can be carried out in any suitable way.

In some embodiments, the sampling step comprises withdrawing a sample of a cell culture from a container containing the cell culture and conveying the sample to the microfluidic device.

In some embodiments, the sample is conveyed via a sampling line to the microfluidic device. In some embodiments, the sampling line comprises an inlet connected to the outlet of the container and an outlet connected to the microfluidic device.

The sampling line can be as described above.

In some embodiments, one or more electric valves, taps and/or flow diverters can control or regulate the flow of sample/liquid between the culture container and the microfluidic device, and/or from the microfluidic device to a waste container.

In some embodiments, for the sampling step, at least one pump is located in-line downstream of the microfluidic device, such that it can draw the sample from the cell culture container to the microfluidic device. The pump can be as described above. In some embodiments, a pump line having a first end is connected to the pump and a second end is connected to the channel outlet. Preferably the pump is a peristaltic pump, providing a sample flow rate of approximately 2 to 17 mL/min. The pump line can be as described above.

In some embodiments, a waste line having a first end can be connected to the pump and a second end can be connected to a waste container. The waste container can collect part or all of the sample, as required.

The waste container can be as described above. The waste line can be as described above.

In some embodiments, the sample can be conveyed to the microfluidic device such that cells of the sample are located within the channel during image capture. Although the sample can flow continuously through the channel during image capture, preferably, sample flow is interrupted and stopped when an image is to be captured.

In some embodiments, the sampling step comprises a pipetting robot adding a sample to the microfluidic device.

In some embodiments, the sampling step comprises a pipetting robot withdrawing a sample of a cell culture and conveying the sample to the microfluidic device.

In some embodiments, the sampling step comprises a user/technician manually adding a sample to the microfluidic device.

In some embodiments, the sampling step comprises a user/technician manually withdrawing a sample of a cell culture and conveying that sample to the microfluidic device.

The imaging system can comprise an imaging device. The imaging system and imaging device can be as described above.

In some embodiments, for the imaging step, the imaging device comprises a microscope, having optics in close proximity of the channel of the microfluidic device, such that cells within the channel can be imaged.

In some embodiments, optics of the imaging device can be adjusted for magnification, focus, contrast and/or resolution, preferably by way of motors, actuators, solenoids and the like.

In some embodiments, a distance between a lens of the optics and the channel can be automatically adjusted such that a focal length of the lens can be adjusted.

In some embodiments, focussing of the lens can be carried out automatically.

In some embodiments, an inverted microscope is used whereby a light source of the microscope is located above the channel and optics/lens of the microscope is located beneath the channel.

In some embodiments, the imaging system or imaging device can comprise a digital camera for capturing still images and/or video recordings of the sample.

In some embodiments, the camera is connected to the imaging device's optics such that images of cells can be captured whilst located within the channel.

In some embodiments, the imaging system can communicate the captured images to the image recognition system in any suitable way, including as described above.

In some embodiments, a single image of the sample is taken. In some embodiments, multiple images of the sample are taken, such as up to 5 images. In some embodiments, a video is taken. Depending on the property of the cell to be determined, a single captured image or an average of multiple captured images (or video image stills) can be used for image recognition.

In some embodiments, the image recognition step comprises executing image recognition software that, when executed, processes captured image data of the cells to determine a property of the cell.

In some embodiments, the imaging system and image recognition system can be in the form of a computational microscope for capturing still images and/or video recordings of the sample within the channel and for carrying out image recognition.

In some embodiments, the image recognition system utilises artificial intelligence (AI)/machine learning as described above.

In some embodiments, captured image data is communicated to a receiver and image recognition software is executed on the receiver. In some embodiments, the receiver or image recognition software utilises artificial intelligence (AI)/machine learning. Preferably the receiver or image recognition software utilises artificial intelligence (AI)/machine learning to determine a property of the cell.

In some embodiments, the image recognition system carries out a first cell detection step based on its ability to detect cell contour. In some embodiments, the image recognition system carries out a subsequent cell property recognition step. In some embodiments, the cell property recognition step comprises a counting step, to count the number of cells present in the image. In some embodiments, the cell property recognition step comprises a cell-feature recognition step whereby: (a) parent and smaller daughter cells in close proximity of each other are recognised; (b) cell surface area and/or cell volume is calculated; (c) cell clumps are recognised; (d) cell colour is recognised; (e) two different cell types (eg. algal and bacterial) are discriminated between; or, (f) the cell species or stain type is identified. Further details for carrying out these steps is as described above.

In some embodiments, a user can request the property of the cell that needs to be determined.

In some embodiments, the image recognition system controls focussing of the imaging device's lens such that the lens automatically scans the depth of the channel and preferentially focuses on cells within the channel.

In some embodiments, the image recognition system causes the imaging device lens to focus on the largest number of cells within the channel.

In some embodiments, the image recognition system causes focussing of the imaging device's lens such that the lens automatically scans the depth of the channel and preferentially focuses on cells within the channel.

In some embodiments, the image recognition system causes the imaging device lens to focus on the largest number of cells within the channel.

In some embodiments, the image recognition system causes the lens to focus through different depths/planes of the channel, until it locates the highest number of cells.

In some embodiments, the image recognition system causes the lens to focus through different depths/planes of the channel, until it locates the highest number of cells, and then uses that same depth/plane for image capture of other subsequent samples added to the channel.

In some embodiments, the method can comprise at least one pre-sampling wetting step, aiding in conveyance of the sample to the microfluidic device. In some embodiments, the method can comprise at least one pre-sampling wetting step, aiding in conveyance of the sample from the cell culture container to the microfluidic device. A pre-wetting step is particularly useful where a small sample quantity is used. The at least one pre-sampling wetting step can be carried out in any suitable way. In some embodiments, the at least one pre-sampling wetting step comprises wetting the microfluidic device and for some embodiments at least part of the sampling line with a wetting agent.

The pre-sampling wetting step can comprise conveying the wetting agent from a container containing the wetting agent to the sampling line and/or to the microfluidic device via a wetting line. The wetting line can have an inlet connected to an outlet of the wetting agent container, and an outlet connected to the sampling line upstream of the microfluidic device or to the microfluidic device itself.

The wetting line can be as described above. The wetting agent container can be as described above.

Any suitable type of wetting agent can be used. In some embodiments, the wetting agent is water or other polar solvent, such as ethyl alcohol.

In some embodiments, for the pre-sampling wetting step, one or more electric valves, taps and/or flow diverters can be used for controlling or regulating the flow of wetting agent/liquid between the wetting agent container and the microfluidic device, as well as from the microfluidic device to a waste container. One or more connectors, fittings etc. can be used for connecting components of the system together.

In some embodiments, for the pre-sampling wetting step, at least one pump can be used for conveying the wetting agent to the microfluidic device and via the sampling line for some embodiments. The pump can be as described above. In some embodiments, at least one pump is located in-line downstream of the microfluidic device, such that it can draw the wetting agent from the wetting agent container to the microfluidic device. Preferably the pump is a peristaltic pump, providing a sample flow rate of approximately 2 to 17 mL/min.

In some embodiments, a pump line having a first end is connected to the pump and a second end is connected to the channel outlet. The pump line can be as described above.

In some embodiments, a waste line having a first end is connected to the pump and a second end is connected to a waste container. The waste container can collect part or all of the wetting agent, if necessary. The waste container can be as described above. The waste line can be as described above.

The method can comprise a sample-cleaning step, comprising cleaning the microfluidic device and for some embodiments also at least part of the sampling line.

This step can remove cells and other culture debris from the conveyance of the sample—particularly a small sample quantity—to the microfluidic device.

This step can flush the microfluidic device and for some embodiments also the sampling line with at least one type of fluid. The fluid can be a gas and/or a liquid.

In some embodiments, the step comprises flushing the microfluidic device and for some embodiments also the sampling line with a gas such as air. In some embodiments, an air pump can be used. Preferably, a pump located downstream of the microfluidic device can displace the sample from the microfluidic device and for some embodiments also the sampling line using air, and for some embodiments also return the sample or most of the sample to the cell culture container.

In some embodiments, the step comprises flushing the microfluidic device and for some embodiments also at least part of the sampling line with a cleaning agent.

Preferably, the step can both flush the microfluidic device and for some embodiments also the sampling line with a gas such as air, and further flush the microfluidic device and for some embodiments also at least part of the sampling line with a cleaning agent.

In some embodiments, a container adapted to contain a cleaning agent, and a cleaning line for conveying the cleaning agent from the cleaning agent container to the sampling line and/or microfluidic device is used.

Any suitable type of cleaning agent can be used. In some embodiments, the cleaning agent is water or other polar solvent, such as ethyl alcohol.

In some embodiments, both the wetting and cleaning agents are water.

The cleaning line can have an inlet connected to an outlet of the cleaning agent container, and an outlet connected to the sampling line upstream of the microfluidic device or to the microfluidic device itself. The cleaning line can be as described above.

In some embodiments, one or more electric valves, taps and/or flow diverters are used for controlling or regulating the flow of cleaning agent/liquid between the cleaning agent container and the microfluidic device, as well as from the microfluidic device to a waste container. One or more connectors, fittings etc. can be used for connecting components of the system together.

In some embodiments, at least one pump is used for conveying the cleaning agent to the microfluidic device and for some embodiments via the sampling line or part thereof. The pump can be as described above. In some embodiments, at least one pump is located in-line downstream of the microfluidic device, such that it can draw the cleaning agent from the cleaning agent container to the microfluidic device. Preferably the pump is a peristaltic pump, providing a sample flow rate of approximately 2 to 17 mL/min.

In some embodiments, a pump line having a first end is connected to the pump and a second end is connected to the channel outlet. The pump line can be as described above.

In some embodiments, a waste line having a first end is connected to the pump and a second end is connected to a waste container. The waste container can collect part or all of the cleaning agent, if necessary. The waste container can be as described above. The waste line can be as described above.

In some embodiments, the imaging and image recognition steps can be automated using a controller. In some embodiments, the imaging, image recognition, and sample-cleaning steps can be automated using a controller. In some embodiments, the sampling, imaging, image recognition, and sample-cleaning steps can be automated using a controller. In some embodiments, the sampling, imaging, image recognition, sample-cleaning, and pre-sampling wetting steps can be automated using a controller.

The controller can be of any suitable construction, including as described above. In some embodiments, the controller utilises artificial intelligence (AI)/machine learning as described above.

The controller can be electrically connected to and control electrical/electronic components of the automated cell analyser, such as the pump/s, valve/s, flow diverter/s, tap/s, solenoid/s, actuator/s, motor/s, imaging device, camera and/or image recognition system.

The controller can comprise a microcontroller electrically connected to the pump/s, valve/s flow diverter/s, actuator/s, motor/s, and imaging device's electricals/electronics etc, for control thereof.

Any suitable cell property can be determined. For example, the property can be one or more of: the degree of cell growth; cell count; cell health; cell morphology; the level of recombinant protein expression by the cell; the level of biomolecule (eg. amino acid, peptide, protein, carbohydrate, sugar, lipid, oil, nucleic acid) expressed by the cell; cell maturity; and, one or more traits of the cell.

According to a fourth aspect of the present invention, there is provided an automated method of determining a cell property, comprising:
(1) at least one sampling step, comprising conveying a sample containing at least one cell to a microfluidic device associated with an imaging system;
(2) at least one imaging step, comprising using the imaging system to capture one or more images of the at least one cell of the sample located within the microfluidic device;
(3) at least one image recognition step, comprising recognising the at least one cell within the one or more captured images of the sample, to thereby determine a property of the cell; and
(4) at least one sample-cleaning step, comprising cleaning the microfluidic device prior to adding a new sample as per step (1), wherein:
steps (3) and (4) need not be carried out in the stated order;
steps (1) to (4) are preferably repeated using new cell samples; and
at least steps (2) to (4) are automated, and optionally step (1) is also automated.

According to a fifth aspect of the present invention, there is provided an automated method of determining a property of a cell, said method comprising the steps of:
(1) adding a cell sample to a microfluidic device;
(2) capturing one or more images of at least one cell of the sample located within the microfluidic device;
(3) carrying out image recognition of the one or more images to thereby determine a property of the at least one cell; and
(4) cleaning the microfluidic device prior to adding a new cell sample to the microfluidic device, wherein:
steps (3) and (4) need not be carried out in the stated order;
steps (1) to (4) are preferably repeated using new cell samples; and
at least steps (2) to (4) are automated.

The cell sample can be as described herein for the other aspects of the invention.

The cell property can be as described herein for the other aspects of the invention.

Step (1) can be carried out using any features of the sampling system or sampling step as described herein.

If using more than one microfluidic device, then features for the methods of the second and third aspects can be as described for the first aspect of the invention.

Step (1) can be carried out: manually by a technician/user; using a pipetting robot; or, using a sampling system or sampling step as described herein.

Preferably step (1) is also automated, as described herein.

Step (2) can be carried out using any features of the imaging system or imaging step as described herein.

Step (3) can be carried out using any features of the image recognition system or image recognition step as described herein.

Step (4) can be carried out using any features of the sample-cleaning system or sample-cleaning steps as described herein.

Automation can be carried out using any features of the controller as described herein.

The method can comprise the step of pre-wetting the microfluidic device, prior to step (1).

Pre-wetting can be carried out using any features of the pre-sampling wetting system or pre-sampling wetting step as described herein.

Preferred embodiments of the invention are described in the following numbered paragraphs.

1. An automated cell analyser for determining a property of a cell, comprising:
a sampling system comprising a microfluidic device for receiving a sample comprising at least one cell;
an imaging system associated with the microfluidic device, adapted to capture one or more images of the at least one cell of the sample located within the microfluidic device;
an image recognition system for recognising the at least one cell within the one or more captured images of the sample, to thereby determine a property of the at least one cell; and
a controller for automating at least the imaging and image recognition systems.

2. The analyser of paragraph 1, wherein the microfluidic device comprises at least one channel in which the sample is received.

3. The analyser of paragraph 2, wherein the imaging system comprises an imaging device comprising:
optics in close proximity of the channel of the microfluidic device such that cells within the channel can be imaged; and
a light source for illuminating any cells within the channel.

4. The analyser of paragraph 3, wherein the imaging device comprises a light and a positioning mechanism for adjusting the distance or position of the light relative to the channel, such that a focal point of the light is adjustable.

5. The analyser of paragraph 3 or paragraph 4, wherein the optics comprise a lens and a lens positioning mechanism for adjusting the distance of the lens relative to the channel.

6. The analyser of paragraph 5, wherein the lens positioning mechanism enables infinite positioning of the lens.

7. The analyser of paragraph 6, wherein positioning and focussing of the lens is carried out automatically.

8. The analyser of any one of paragraphs 3 to 7, wherein the imaging device comprises an inverted light microscope whereby the light source is located above the channel and the optics are located beneath the channel.

9. The analyser of any one of paragraphs 3 to 8, wherein the imaging system or the imaging device comprises a digital camera for capturing image data in the form of a still image and/or video recording of the sample.

10. The analyser of paragraph 9, wherein the image recognition system comprises image recognition software that, when executed, processes captured image data of the cell to determine the property of the at least one cell.

11. The analyser of paragraph 10, wherein the image recognition system utilises artificial intelligence/machine learning to determine the property of the cell.

12. The analyser of paragraph 10 or paragraph 11, wherein the image recognition system carries out a first cell detection step based on the detection of cell contour.

13. The analyser of paragraph 12, wherein the image recognition system carries out a subsequent cell property recognition step.

14. The analyser of paragraph 13, wherein the cell property recognition step comprises a counting step, to count the number of cells present in the image.

15. The analyser of paragraph 13 or paragraph 14, wherein the cell property recognition step comprises a cell-feature recognition step whereby: (a) parent and smaller daughter cells in close proximity of each other are recognised; (b) cell surface area and/or cell volume is calculated; (c) cell clumps are recognised; (d) cell colour is recognised; (e) two different cell types are discriminated between; or, (f) the cell species or stain type is identified.

16. The analyser of any one of paragraphs 5 to 15 when dependent on paragraph 5, wherein the image recognition system controls positioning and focussing of the lens such that the lens automatically scans the depth of the channel and preferentially focuses on any cells within the channel.

17. The analyser of paragraph 16, wherein focussing and scanning of the lens is carried out using artificial intelligence (AI)/machine learning.

18. The analyser of paragraph 17, wherein the image recognition system causes the lens to focus on the largest number of cells within the channel.

19. The analyser of any one of paragraphs 10 to 18 when dependent on paragraph 10, wherein captured image data is communicated to a receiver and the image recognition software is executed on the receiver.

20. The analyser of paragraph 19, wherein the receiver comprises a standalone computer, a computer network, a website interface, a smart phone, or other electronic device.

21. The analyser of any one of paragraphs 2 to 20, wherein the channel has a depth of about 20-100 microns, preferably about 30-50 microns, more preferably about 30 microns.

22. The analyser of any one of paragraphs 2 to 21, wherein the microfluidic device is in the form of a chip comprising a chip body and the at least one channel extending along and/or within the chip body.

23. The analyser of any one of paragraphs 1 to 22, wherein the at least one cell is a unicellular organism, a prokaryotic cell, a eukaryotic cell, or a separate cultured cell of a multi-cellular organism.

24. The analyser of paragraph 23, wherein the at least one cell is a microalgal cell.

25. The analyser of any one of paragraphs 1 to 24, wherein:
(a) the sample is a sample of a cell culture, and the sampling system is able to withdraw and convey the sample of the cell culture to the microfluidic device;
(b) the sampling system comprises a pipetting robot for adding the sample to the microfluidic device; or
(c) the sampling system enables a user to manually add the sample to the microfluidic device.

26. The analyser of paragraph 25, wherein said sampling system of (a) comprises the microfluidic device, and a sampling line for conveying the sample between a container adapted to contain the cell culture and the microfluidic device.

27. The analyser of paragraph 26, wherein the sampling system optionally comprises the container adapted to contain the cell culture and the sampling line comprises an inlet connected to an outlet of the cell culture container and an outlet connected to the microfluidic device.

28. The analyser of paragraph 27, wherein the sampling system comprises the cell culture container.

29. The analyser of any one of paragraphs 26 to 28, wherein the sampling system comprises at least one pump for causing flow of the sample and conveying the sample to the microfluidic device.

30. The analyser of paragraph 29, wherein the sampling system comprises a waste container and the pump is capable of further conveying the sample from the microfluidic device to the waste container.

31. The analyser of paragraph 29 or paragraph 30, wherein sample flow is interrupted and stopped within the channel at the time an image is to be captured.

32. The analyser of any one of paragraphs 26 to 31, wherein the automated cell analyser comprises a pre-sampling wetting system for wetting the microfluidic device and optionally the sampling line or part thereof prior to sampling.

33. The analyser of paragraph 32, wherein the pre-sampling wetting system wets the microfluidic device and the sampling line or part thereof, prior to sampling and conveying of the sample.

34. The analyser of paragraph 32 or 33, wherein the pre-sampling wetting system comprises a container adapted to contain a wetting agent, and a wetting line for conveying the wetting agent from the wetting agent container to the sampling line and/or microfluidic device.

35. The analyser of paragraph 34, wherein the wetting agent is water or other polar solvent, such as ethyl alcohol.

36. The analyser of paragraph 34 or paragraph 35, wherein the pre-sampling wetting system comprises at least one pump for conveying the wetting agent to the microfluidic device, optionally via the sampling line.

37. The analyser of any one of paragraphs 26 to 36 when dependent on paragraph 26, wherein the analyser comprises a sample-cleaning system for cleaning the microfluidic device and optionally also the sampling line or part thereof, after the imaging step.

38. The analyser of paragraph 37, wherein the sample-cleaning system flushes the microfluidic device and optionally also the sampling line or part thereof with at least one type of fluid.

39. The analyser of paragraph 38, wherein the sample-cleaning system flushes the microfluidic device and optionally also the sampling line with a gas such as air.

40. The analyser of paragraph 39, wherein the analyser comprises at least one pump located downstream of the microfluidic device for displacing the sample from the microfluidic device and optionally also from the sampling line using air.

41. The analyser of any one of paragraphs 38 to 40, wherein the sample-cleaning system flushes the microfluidic device and optionally also the sampling line with a cleaning agent.

42. The analyser of any one of paragraphs 38 to 40, wherein the sample-cleaning system both flushes the microfluidic device and optionally also the sampling line with a gas such as air, and further flushes the microfluidic device and optionally also at least part of the sampling line with a cleaning agent.

43. The analyser of paragraph 42, wherein the cleaning agent is water or other polar solvent, such as ethyl alcohol.

44. The analyser of any one of paragraphs 1 to 43, wherein the controller utilises artificial intelligence (AI)/machine learning and automates: the imaging, image recognition, and sampling systems.

45. The analyser of any one of paragraphs 37 to 44 when dependent on paragraph 37, wherein the controller automates: the imaging, image recognition, sampling, and sample-cleaning systems.

46. The analyser of any one of paragraph 32 to 44 when dependent on paragraph 32, wherein the controller automates: the imaging, image recognition, sampling, sample-cleaning, and pre-sampling wetting systems.

47. The analyser of any one of paragraphs 1 to 46, wherein the property is one or more of: the degree of cell growth; cell count; cell health; cell morphology; cell/organism species identification; cell/organism strain identification; a level of recombinant protein expression by the cell; a level of a biomolecule expressed by the cell; cell maturity; and, one or more traits of the cell.

48. An automated method of determining a cell property, comprising:
at least one sampling step, comprising conveying a sample containing at least one cell to a microfluidic device associated with an imaging system;
at least one imaging step, comprising using the imaging system to capture one or more images of the at least one cell of the sample located within the microfluidic device; and
at least one image recognition step, comprising recognising the at least one cell within the one or more captured images of the sample, to thereby determine a property of the cell,
wherein at least the imaging and image recognition steps are automated.

49. The method of paragraph 48, wherein:
(a) the sampling step comprises withdrawing a sample of a cell culture from a container containing the cell culture and conveying the sample to the microfluidic device;
(b) the sampling step comprises a pipetting robot adding a sample to the microfluidic device;
(c) the sampling step comprises a pipetting robot withdrawing a sample of a cell culture and conveying the sample to the microfluidic device;
(d) the sampling step comprises a user manually adding a sample to the microfluidic device; or
(e) the sampling step comprises a user manually withdrawing a sample of a cell culture and conveying that sample to the microfluidic device.

50. The method of paragraph 49, wherein for sampling step (a) the sample is conveyed via a sampling line to the microfluidic device.

51. The method of paragraph 50, wherein the sampling line comprises an inlet connected to an outlet of the container and an outlet connected to the microfluidic device.

52. The method of paragraph 51, wherein at least one pump is located in-line downstream of the microfluidic device, such that the pump can draw the sample from the cell culture container to the microfluidic device.

53. The method of paragraph 52, wherein a waste line having a first end is connected to the pump and a second end of the waste line is connected to a waste container.

54. The method of any one of paragraphs 49 to 53, wherein the sample is conveyed to the microfluidic device such that cells of the sample are located within the channel during image capture.

55. The method of paragraph 54, wherein sample flow is interrupted and stopped when an image is to be captured.

56. The method of any one of paragraphs 48 to 55, wherein the imaging system comprises an imaging device comprising a microscope, having optics in close proximity of the channel of the microfluidic device, such that cells within the channel are imaged.

57. The method of paragraph 56, wherein the optics of the imaging device are adjustable for magnification, focus, contrast and/or resolution.

58. The method of paragraph 57, wherein a distance between a lens of the optics and the channel is automatically adjusted such that a focal length of the lens is adjustable.

59. The method of paragraph 58, wherein focussing of the lens is carried out automatically.

60. The method of any one of paragraphs 56 to 59, wherein the microscope is an inverted microscope whereby a light source of the microscope is located above the channel and optics/lens of the microscope are located beneath the channel.

61. The method of any one of paragraphs 56 to 60, wherein the imaging system or imaging device comprises a digital camera for capturing still images and/or video recordings of the sample.

62. The method of paragraph 61, wherein the camera is connected to the imaging device's optics such that images of cells are captured whilst located within the channel.

63. The method of paragraph 61 or paragraph 62, wherein a single image of the sample is taken, multiple images of the sample are taken, or a video is taken for use in the image recognition step.

64. The method of paragraph 63, wherein the image recognition step comprises executing image recognition software that, when executed, processes captured image data of the cells to determine the property of the cell.

65. The method of paragraph 64, wherein the image recognition system utilises artificial intelligence (AI)/machine learning.

66. The method of paragraph 64 or paragraph 65, wherein captured image data is communicated to a receiver and image recognition software is executed on the receiver.

67. The method of paragraph 64, 65 or 66, wherein the receiver or image recognition software utilises artificial intelligence (AI)/machine learning to determine the property of the cell.

68. The method of any one of paragraphs 48 to 67, wherein the image recognition system carries out a first cell detection step based on an ability to detect cell contour.

69. The method of paragraph 68, wherein the image recognition system carries out a subsequent cell property recognition step.

70. The method of paragraph 69, wherein the cell property recognition step comprises a counting step, to count the number of cells present in the image.

71. The method of paragraph 69, wherein the cell property recognition step comprises a cell-feature recognition step whereby: (a) parent and smaller daughter cells in close proximity of each other are recognised; (b) cell surface area and/or cell volume is calculated; (c) cell clumps are recognised; (d) cell colour is recognised; (e) two different cell types are discriminated between; or, (f) the cell species or stain type is identified.

72. The method of any one of paragraphs 48 to 71, wherein a user requests the property of the cell that needs to be determined.

73. The method of any one of paragraphs 58 to 72 when dependent on paragraph 58, wherein the image recognition system controls focussing of the imaging device's lens such that the lens automatically scans the depth of the channel and preferentially focuses on cells within the channel.

74. The method of any one of paragraphs 58 to 73 when dependent on paragraph 58, wherein the image recognition system causes the imaging device lens to focus on the largest number of cells within the channel.

75. The method of any one of paragraphs 58 to 73 when dependent on paragraph 58, wherein the image recognition system causes the lens to focus through different depths/planes of the channel, until it locates the highest number of cells, and then uses that same depth/plane for image capture of other subsequent samples added to the channel.

76. The method of any one of paragraphs 48 to 75, wherein the method comprises at least one pre-sampling wetting step, aiding in conveyance of the sample to the microfluidic device.

77. The method of paragraph 76, wherein the pre-sampling wetting step aids in conveyance of the sample from a cell culture container to the microfluidic device.

78. The method of paragraph 76 or paragraph 77, wherein the pre-sampling wetting step comprises wetting the microfluidic device and optionally at least part of the sampling line with a wetting agent.

79. The method of paragraph 78, wherein the pre-sampling wetting step comprises conveying the wetting agent from a container containing the wetting agent to the sampling line and/or to the microfluidic device via a wetting line.

80. The method of paragraph 79, wherein the wetting line has an inlet connected to an outlet of the wetting agent container, and an outlet connected to the sampling line upstream of the microfluidic device or to the microfluidic device itself.

81. The method of paragraph 79 or paragraph 80, wherein the wetting agent is water or other polar solvent, such as ethyl alcohol.

82. The method of any one of paragraphs 78 to 81, wherein for the pre-sampling wetting step, at least one pump is used for conveying the wetting agent to the microfluidic device and optionally via the sampling line.

83. The method of paragraph 82, wherein the at least one pump is located in-line downstream of the microfluidic device, such that the pump draws the wetting agent from the wetting agent container to the microfluidic device.

84. The method of paragraph 83, wherein a pump line having a first end is connected to the pump and a second end is connected to an outlet of the channel.

85. The method of paragraph 84, wherein a waste line having a first end is connected to the pump and a second end is connected to a waste container.

86. The method of any one of paragraphs 50 to 85 when dependent on paragraph 50, further comprising a sample-cleaning step, comprising cleaning the microfluidic device and optionally at least part of the sampling line.

87. The method of paragraph 86, wherein the sample-cleaning step comprises flushing the microfluidic device and optionally the sampling line with at least one type of fluid.

88. The method of paragraph 87, wherein the sample-cleaning step comprises flushing the microfluidic device and optionally the sampling line with a gas such as air.

89. The method of paragraph 88, wherein at least one pump located downstream of the microfluidic device displaces the sample from the microfluidic device and optionally the sampling line using air.

90. The method of paragraph 87, wherein the sample-cleaning step comprises flushing the microfluidic device and optionally at least part of the sampling line with a cleaning agent.

91. The method of paragraph 90, wherein a container adapted to contain the cleaning agent, and a cleaning line for conveying the cleaning agent from the cleaning agent container to the sampling line and/or microfluidic device is used for flushing.

92. The method of paragraph 91, wherein the cleaning line has an inlet connected to an outlet of the cleaning agent container, and an outlet connected to the sampling line upstream of the microfluidic device or to the microfluidic device itself.

93. The method of any one of paragraphs to 92, wherein the cleaning agent is water or other polar solvent, such as ethyl alcohol.

94. The method of any one of paragraphs 90 to 93, wherein at least one pump is used for conveying the cleaning agent to the microfluidic device and optionally via the sampling line or part thereof.

95. The method of paragraph 94, wherein the at least one pump is located in-line downstream of the microfluidic device, such that the pump can draw the cleaning agent from the cleaning agent container to the microfluidic device.

96 The method of paragraph 95, wherein a pump line having a first end is connected to the pump and a second end is connected to an outlet of the channel.

97. The method of paragraph 96, wherein a waste line having a first end is connected to the pump and a second end is connected to a waste container.

98. The method of any one of paragraphs 87 to 97, wherein the sample-cleaning step comprises both flushing the microfluidic device and optionally the sampling line with a gas such as air, and further flushing the microfluidic device and optionally at least part of the sampling line with a cleaning agent.

99. The method of any one of paragraphs 48 to 98, wherein the imaging and image recognition steps are automated using a controller.

100. The method of any one of paragraphs 86 to 98 when dependent on paragraph 86, wherein the imaging, image recognition, and sample-cleaning steps are automated using a controller.

101. The method of any one of paragraphs 86 to 98 when dependent on paragraph 86, wherein the sampling, imaging, image recognition, and sample-cleaning steps are automated using a controller.

102. The method of any one of paragraphs 76 to 98 when dependent on paragraph 76, wherein the sampling, imaging, image recognition, sample-cleaning, and pre-sampling wetting steps are automated using a controller.

103. The method of paragraph 102, wherein the controller utilises artificial intelligence (AI)/machine learning.

104. The method of any one of paragraphs 48 to 103, wherein the property is one or more of: the degree of cell growth; cell count; cell health; cell morphology; the level of recombinant protein expression by the cell; the level of biomolecule expressed by the cell; cell maturity; and, one or more traits of the cell.

105. The method of any one of paragraphs 48 to 104, when carried out using the analyser of any one of paragraphs 1 to 47.

106. An automated method of determining a cell property, comprising:
(1) at least one sampling step, comprising conveying a sample containing at least one cell to a microfluidic device associated with an imaging system;
(2) at least one imaging step, comprising using the imaging system to capture one or more images of the at least one cell of the sample located within the microfluidic device;
(3) at least one image recognition step, comprising recognising the at least one cell within the one or more captured images of the sample, to thereby determine a property of the cell; and
(4) at least one sample-cleaning step, comprising cleaning the microfluidic device prior to adding a new sample as per step (1), wherein:
steps (3) and (4) need not be carried out in the stated order;
steps (1) to (4) are preferably repeated using new cell samples; and
at least steps (2) to (4) are automated, and optionally step (1) is also automated.

107. The method of paragraph 106, wherein steps (1) to (4) are repeated using new cell samples.

108. The method of paragraph 106 or paragraph 107, wherein step (1) is also automated.

109. An automated method of determining a property of a cell, said method comprising the steps of:
(1) adding a cell sample to a microfluidic device;
(2) capturing one or more images of at least one cell of the sample located within the microfluidic device;
(3) carrying out image recognition of the one or more images to thereby determine a property of the at least one cell; and
(4) cleaning the microfluidic device prior to adding a new cell sample to the microfluidic device, wherein:
steps (3) and (4) need not be carried out in the stated order;
steps (1) to (4) are preferably repeated using new cell samples; and
at least steps (2) to (4) are automated.

110. The method of paragraph 109, wherein steps (1) to (4) are repeated using new cell samples.

111. The method of any one of paragraphs 106 to 110, wherein the method comprises the step of pre-wetting the microfluidic device, prior to step (1).

112. The method of any one of paragraphs 48 to 111, wherein the at least one cell is a unicellular organism, a prokaryotic cell, a eukaryotic cell, or a separate cultured cell of a multi-cellular organism.

113. The method of paragraph 112, wherein the at least one cell is a microalgal cell.

114. The method of any one of paragraphs 48 to 113, for use or when used in the culturing of cells or microorganisms.

115. The method of paragraph 114, wherein the cells are microalgae.

Further preferred embodiments of the invention are described in the following numbered paragraphs.

1. An automated cell analyser for determining a property of a cell, comprising:
a sampling system for sampling a cell culture, comprising a microfluidic device, a sampling line for conveying a cell culture sample from a container adapted to contain the cell culture to a channel of the microfluidic device, and at least one pump located downstream of and in-line with the microfluidic device that moves the sample from the cell culture container to the channel, wherein the sample comprises at least one cell;
an imaging system associated with the microfluidic device, adapted to capture one or more images of the at least one cell of the sample located within the channel;
an image recognition system for recognising the at least one cell within the one or more captured images of the sample, to thereby determine a property of the at least one cell, wherein the image recognition system utilises artificial intelligence/machine learning to determine the property of the at least one cell, and wherein the image recognition system carries out a first cell detection step based on the detection of cell contour, and a subsequent cell property recognition step;
a sample-cleaning system for cleaning the channel and the sampling line or part thereof, comprising at least one pump located downstream of and in-line with the microfluidic device that is operable to displace the sample from the microfluidic device and return the sample or most of the sample to the cell culture container, and is operable to convey a cleaning agent of the sample-cleaning system to the channel and the sampling line or part thereof; and
a controller for automating the sampling, imaging, image recognition and sample-cleaning systems.

2. The analyser of paragraph 1, wherein the automated cell analyser comprises an automated pre-sampling wetting system for wetting the channel and the sampling line or part thereof prior to sampling of the cell culture, comprising at least one pump located downstream of and in-line with the microfluidic device that conveys a wetting agent of the pre-sampling wetting system to the channel and the sampling line of part thereof.

3. An automated method of determining a cell property, comprising:
(1) at least one sampling step, comprising withdrawing a sample of a cell culture from a container containing the cell culture and conveying the sample via a sampling line to a channel of a microfluidic device that is associated with an imaging system, wherein at least one pump located downstream of and in-line with the microfluidic device moves the sample from the cell culture container to the channel, and wherein the sample contains at least one cell;
(2) at least one imaging step, comprising using the imaging system to capture one or more images of the at least one cell of the sample located within the channel;
(3) at least one image recognition step, comprising recognising the at least one cell within the one or more captured images of the sample, to thereby determine a property of the cell, wherein the at least one image recognition step utilises artificial intelligence (AI)/machine learning to determine the property of the cell, wherein the image recognition system carries out a first cell detection step based on the detection of cell contour, and a subsequent cell property recognition step; and
(4) at least one sample-cleaning step, comprising cleaning the channel and at least part of the sampling line, wherein at least one pump located downstream of and in-line with the microfluidic device is operable to displace the sample from the microfluidic device and return the sample or most of the sample to the cell culture container, and is operable to convey a cleaning agent to the channel and the sampling line or part thereof, wherein:
steps (3) and (4) need not be carried out in the stated order;
steps (1) to (4) are repeated using new cell culture samples; and
steps (1) to (4) are automated using a controller.

4. The method of paragraph 3, further comprising:
at least one pre-sampling wetting step comprising wetting the channel and at least part of the sampling line with a wetting agent, wherein at least one pump located downstream of and in-line with the microfluidic device conveys the wetting agent to the channel of the microfluidic device and the sampling line of part thereof, wherein the pre-sampling wetting step is automated using the controller.

5. The analyser of paragraph 1 or paragraph 2, or the method of paragraph 3 or paragraph 4, wherein a waste line having a first end is connected to each said at least one pump and a second end is connected to a waste container.

6. The analyser or method of paragraph 5, wherein the at least one pumps are one and the same.

7. The analyser of any one of paragraphs 1, 2, 5 and 6, or the method of any one of paragraphs 3 to 6, wherein the sample-cleaning step comprises flushing the channel and at least part of the sampling line with both the cleaning agent and a gas such as air.

8. The analyser of any one of paragraphs 1, 2 and 5 to 7, or the method of any one of paragraphs 3 to 7, wherein the property of the cell is one or more of: the degree of cell growth; cell count; cell health; cell morphology; the level of recombinant protein expression by the cell; the level of biomolecule expressed by the cell; cell maturity; and, one or more traits of the cell.

9. The analyser of any one of paragraphs 1, 2 and 5 to 8, or the method of any one of paragraphs 3 to 8, wherein the cell property recognition step comprises: a counting step, to count the number of cells present in the one or more captured images; or, a cell-feature recognition step whereby: (a) parent and smaller daughter cells in close proximity of each other are recognised; (b) cell surface area and/or cell volume is calculated; (c) cell clumps are recognised; (d) cell colour is recognised; (e) two different cell types are discriminated between; or, (f) the cell species or stain type is identified.

10. The analyser of any one of paragraphs 1, 2 and 5 to 9, or the method of any one of paragraphs 3 to 9, wherein sample flow is interrupted and stopped within the channel when an image is to be captured.

11. The analyser of any one of paragraphs 1, 2 and 5 to 10, or the method of any one of paragraphs 3 to 10, wherein the imaging system comprises an imaging device comprising:
optics in close proximity of the channel such that any cell within the channel can be imaged; and
a light source for illuminating any cells within the channel.

12. The analyser or method of paragraph 11, wherein the optics comprise a lens and a lens positioning mechanism for adjusting the distance of the lens relative to the channel, wherein positioning and focussing of the lens is carried out automatically.

13. The analyser or method of paragraph 12, wherein the image recognition system controls positioning and focussing of the lens such that the lens automatically scans the depth of the channel and focuses on any cells within the channel.

14. The analyser or method of paragraph 12 or paragraph 13, wherein the image recognition system causes the lens to focus on the largest number of cells within the channel.

15. The analyser or method of any one of paragraphs 12 to 14, wherein the image recognition system causes the lens to focus through different depths or planes of the channel, until it locates the highest number of cells, and then uses that same depth or plane for image capture of other subsequent samples added to the channel.

16. The analyser or method of any one of paragraphs 11 to 15, wherein the imaging device comprises an inverted light microscope whereby the light source of the inverted light microscope is located above the channel and the optics of the inverted light microscope are located beneath the channel.

17. The analyser of any one of paragraphs 1, 2 and 5 to 16, or the method of any one of paragraphs 3 to 16, wherein the at least one cell is a unicellular organism, a prokaryotic cell, a eukaryotic cell, or a separate cultured cell of a multi-cellular organism.

18. The analyser or method of paragraph 17, wherein the at least one cell is a microalgal cell.

19. The analyser of any one of paragraphs 1, 2 and 5 to 18, or the method of any one of paragraphs 5 to 18, when used for periodically sampling a microalgae cell culture, so as to automatically optimise growth of microalgae within that cell culture.

Any of the features described herein can be combined in any combination with any one or more of the other features described herein within the scope of the invention. The features described with respect to one aspect also apply where applicable to all other aspects of the invention. Furthermore, different combinations of described features are herein described and claimed even when not expressly stated.

The reference to any prior art in this specification is not and should not be taken as an acknowledgement or any form of suggestion that the prior art forms part of the common general knowledge.

### BRIEF DESCRIPTION OF THE FIGURES

In order that the present invention can be more readily understood reference will now be made to the accompanying drawings which illustrate a preferred embodiment of the invention and wherein:

FIG. 1 shows part of an automated cell analyser (omitting fluid circuitry) for determining a property of a cell, such as microalgae growth, as well as steps of an automated method using that analyser, according to an embodiment of the present invention.

FIG. 2 shows fluid schematics for the automated cell analyser of FIG. 1, according to an embodiment of the present invention.

FIG. 3 is the same as FIG. 2, except showing a pre-sampling wetting step.

FIG. 4 is the same as FIG. 3, except showing a microalgae culture sampling step.

FIG. 5 is the same as FIG. 4, except showing a subsequent imaging step.

FIG. 6 is the same as FIG. 5, except showing a subsequent first cleaning step.

FIG. 7 is the same as FIG. 6, except showing a subsequent second cleaning step.

FIG. 8 is the same as FIG. 6, except highlighting which lines are cleaned by the first and second cleaning steps.

FIG. 9 shows images of Chlamydomonas reinhardtii algal cells of four different samples, each photographed within a 30-micron channel of a microfluidic device, at 20× microscope magnification.

FIG. 10 shows images of Porphyridium cruentum algal cells of four different samples, each photographed within a 30-micron channel of a microfluidic device, at 20× microscope magnification.

FIG. 11 is an image showing detection of microalgae cell contour at 40× microscope magnification.

FIG. 12 is an image showing counting of microalgae cells at 40× microscope magnification.

FIG. 12a shows a magnified region of FIG. 12.

FIG. 13 is an image showing recognition of and discrimination between microalgal and bacterial cells, at 40× microscope magnification.

FIG. 13a shows a magnified region of FIG. 13.

FIG. 14 is an image showing the calculation of cell surface area and/or cell volume, at 40× microscope magnification.

FIG. 15 is an image showing recognition of cell clumps, at 40× microscope magnification.

FIG. 16 is a flowchart, showing cell detection performed with YOLO and feature detection using U-net.

FIG. 16a shows a magnified part of the flowchart of FIG. 16.

FIG. 16b shows a magnified part of the flowchart of FIG. 16.

FIG. 16c shows a magnified part of the flowchart of FIG. 16.

In the figures like reference numerals refer to like parts.

### DESCRIPTION OF THE PREFERRED EMBODIMENTS

Preferred features, embodiments and variations of the invention may be discerned from this section, which provides sufficient information for those skilled in the art to perform the invention. This section is not to be regarded as limiting the scope of any preceding section in any way.

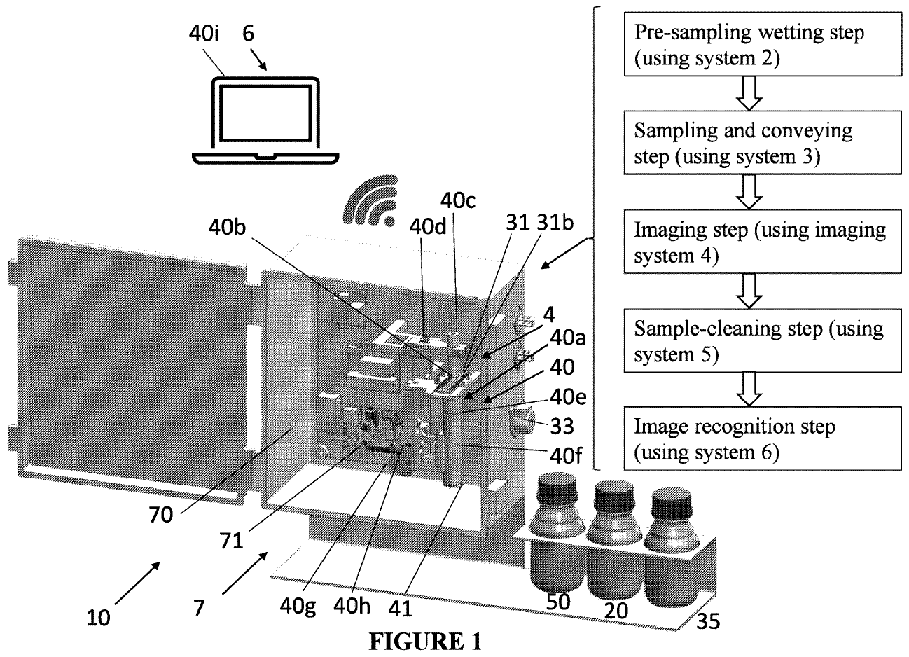

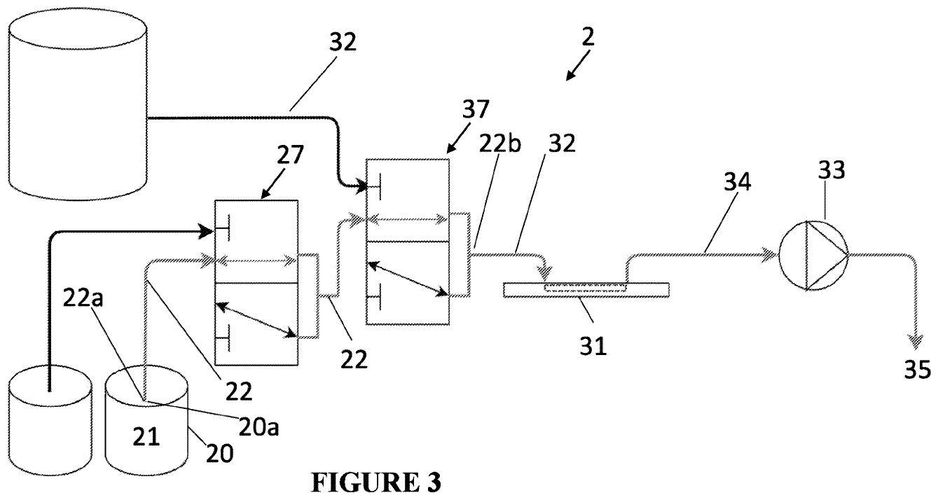

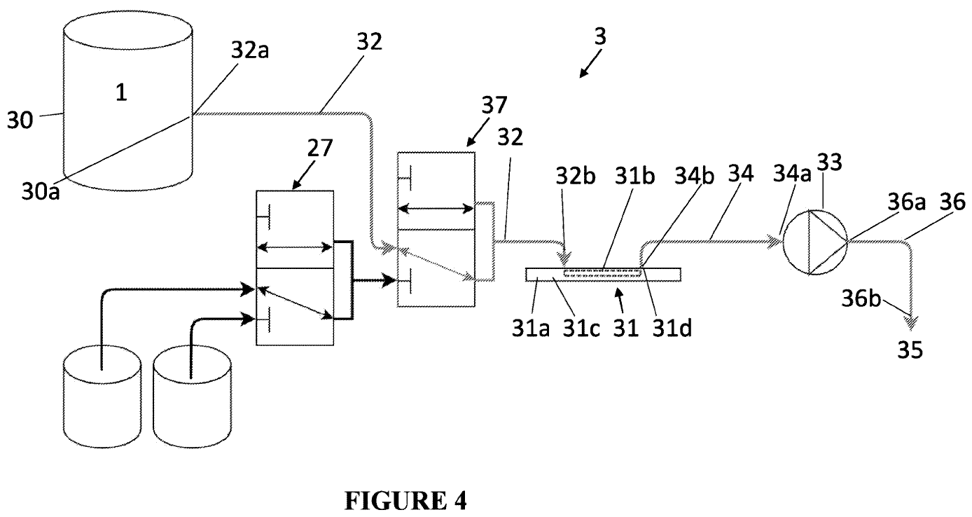

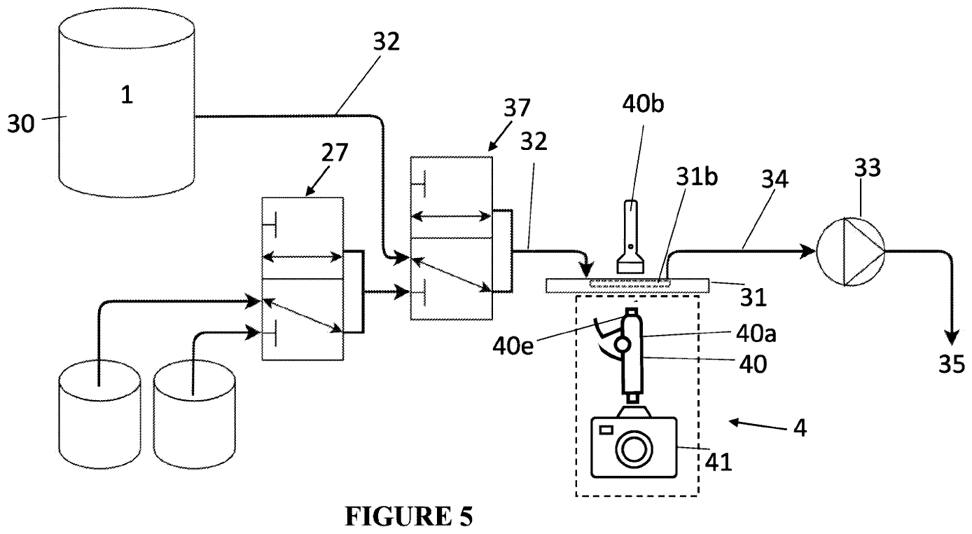

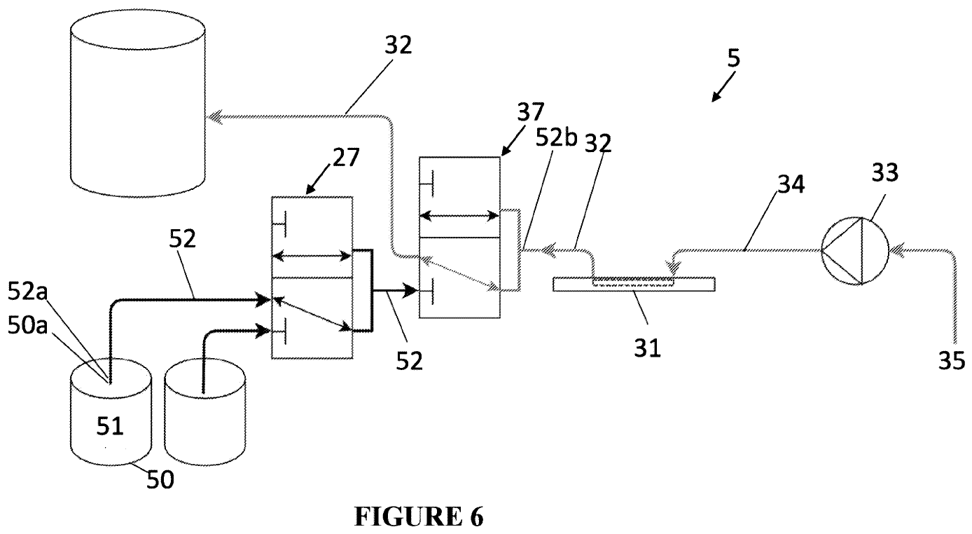

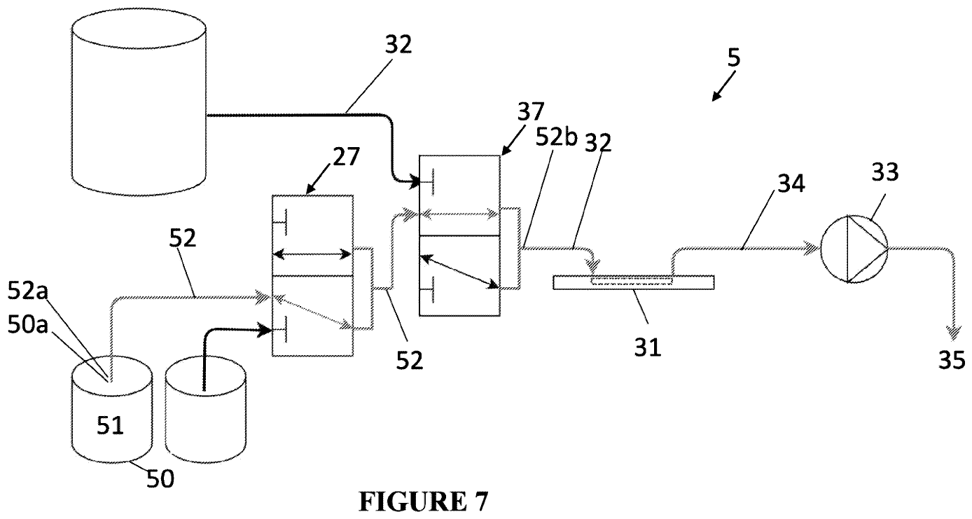

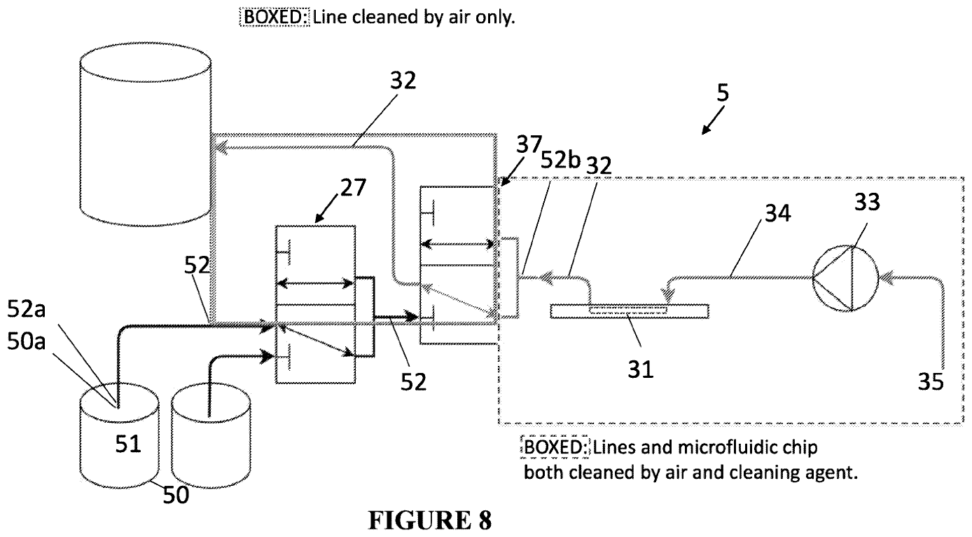

FIGS. 1-8 show part of an automated cell analyser 10 for determining a property of a cell, such as microalgae growth. The automated cell analyser 10 includes a pre-sampling wetting system 2 (see FIG. 3), a sampling system 3 (see FIG. 4), an imaging system 4 (see FIGS. 1 and 5), a sample-cleaning system 5 (see FIGS. 6-8), an image recognition system 6, and a controller 7 (see FIG. 1).

As seen in FIGS. 1, 2 and 4, the sampling system 3 is for withdrawing and conveying a sample of a microalgae culture 1 (or other type of substantially unicellular prokaryotic or eukaryotic cell culture). The sampling system 3 includes a container 30 (200L bioreactor) adapted to contain the microalgae culture 1, a microfluidic device 31, a sampling line 32 for conveying the sample between the container 30 and the microfluidic device 31, and various electric valves and flow diverters/3-way valves 37.

The sampling line 32 includes an inlet 32a connected to an outlet 30a of the microalgae culture container 30 and an outlet 32b connected to the microfluidic device 31. The sampling line 32 comprises lengths of platinum cured silicone tubing connected together, having an internal diameter of approximately 0.6 mm.

The microfluidic device 31 is a chip comprising a chip body 31a and a channel 31b extending within the chip body 31a. The channel 31b has an inlet 31c and an outlet 31d. The channel 31b has a depth of 30 microns. The channel inlet 31c is connected to the outlet 32b of the sampling line 32.

The sampling system 3 further includes a pump 33 located in-line downstream of the microfluidic device 31, such that the pump 33 can draw the sample from the microalgae culture container 30 to the microfluidic device 31. The pump 33 is a peristaltic pump, providing a sample flow rate of approximately 2 to 17 mL/min.

The sampling system 3 also includes a pump line 34 having a first end 34a connected to the pump 33 and a second end 34b connected to the channel outlet 31d. The pump line 34 comprises a length of platinum cured silicone tubing, having an internal diameter of approximately 0.6-1 mm.

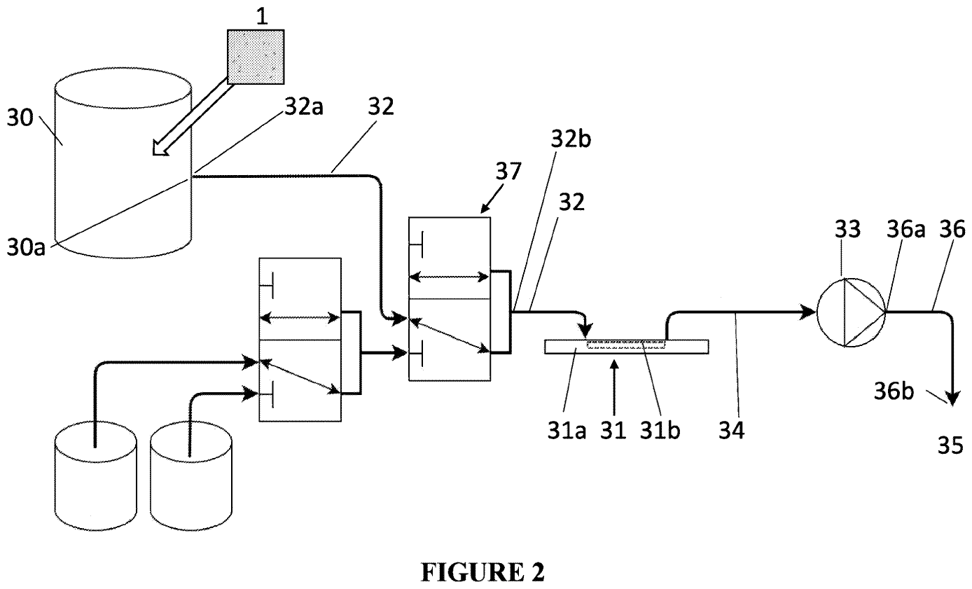

The sampling system 3 further includes a waste container 35 (see FIG. 2) and a waste line 36 having a first end 36a connected to the pump 33 and a second end 36b connected to the waste container 35. The waste container 35 is a bottle. The waste container 35 can collect part or all of the sample, as required.

The waste line 36 comprises a length of platinum cured silicone tubing, having an internal diameter of approximately 1 mm.

The various electric valves and flow diverters/3-way valves 37 control the flow of sample/liquid between the culture container 30 and the microfluidic device 31, as well as from the microfluidic device 31 to the waste container 35.

The sampling system 3 can convey the sample to the microfluidic device 31 such that microalgal cells 1 of the sample are present within the channel 31b for image capture.

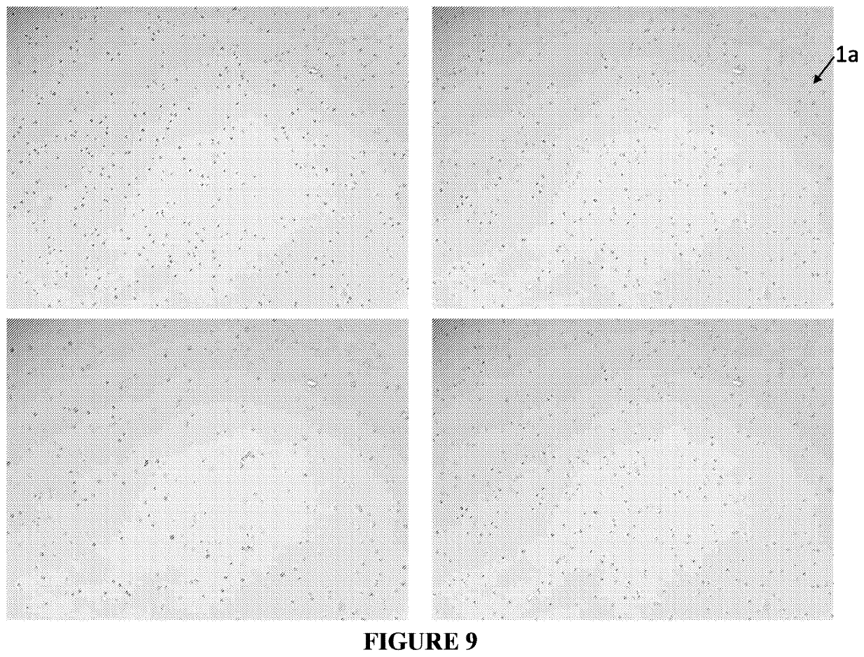

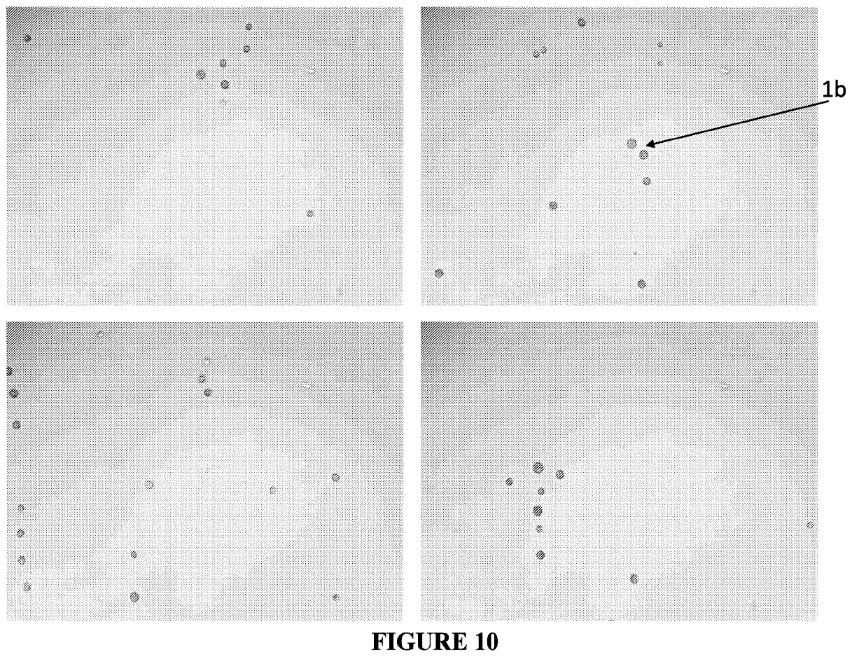

As seen in FIGS. 1 and 5, the imaging system 4 comprises an imaging device 4 associated with the microfluidic device 31, and is able to capture images of microalgal cells 1a, 1b of the sample located within the microfluidic device 31. Images of Chlamydomonas reinhardtii microalgal cells 1a are shown in FIG. 9. Images of Porphyridium cruentum microalgal cells 1b are shown in FIG. 10.

The imaging device 4 includes an inverted light microscope 40 and camera 41. The microscope 40 has both optics 40a and electronics. The camera 41 has both optics and electronics.

The microscope optics 40a are in close proximity of the channel 31b of the microfluidic device 31, such that algal cells 1a, 1b within the channel 31b can be clearly imaged.

The microscope 40 includes an LED light source 40b for illuminating microalgal cells within the channel 31b, and a light housing 40c. The light source 40b includes a positioning mechanism 40d for adjusting the distance/position of the light source 40b relative to the channel 31b, such that a focal point of the light can be adjusted. The positioning mechanism 40d is a rack and pinion mechanism indirectly connected to the light housing 40c.

The microscope optics 40a include a lens 40e located within a lens housing 40f. The optics 40a include a positioning mechanism 40g for adjusting the distance of the lens 40e relative to the channel 31b, such that a focal length of the lens 40e can be adjusted. The positioning mechanism 40g is motorised/electronic for automatic adjustment, and includes a stepper motor 40h and endless belt arrangement (not shown) connected to the lens 40e for moving the lens 40e substantially vertically relative to the channel 31b so that the lens 40e can focus on different channel depths/planes.

The camera 41 is connected to the lens housing 40c below the lens 40e.

As seen in FIG. 1, the imaging system 4 communicates the captured images to the image recognition system 6 wirelessly (Wi-Fi) using a wireless transceiver.

Depending on the property of the microalgae growth to be determined, a single captured image or an average of multiple captured images (eg. 5 images) can be used for image recognition.

The image recognition system 6 includes image recognition software that, when executed on a receiver 40i (eg. computer, computer network, a website interface, smart phone or other electronic device), processes captured image data of the microalgal cells to determine a property of the microalgae growth.

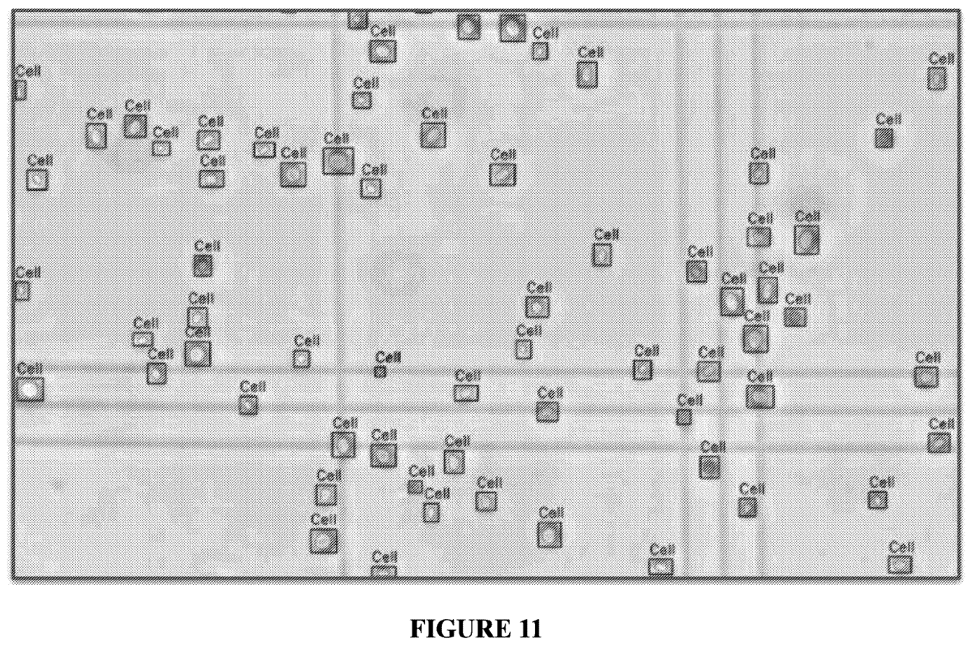

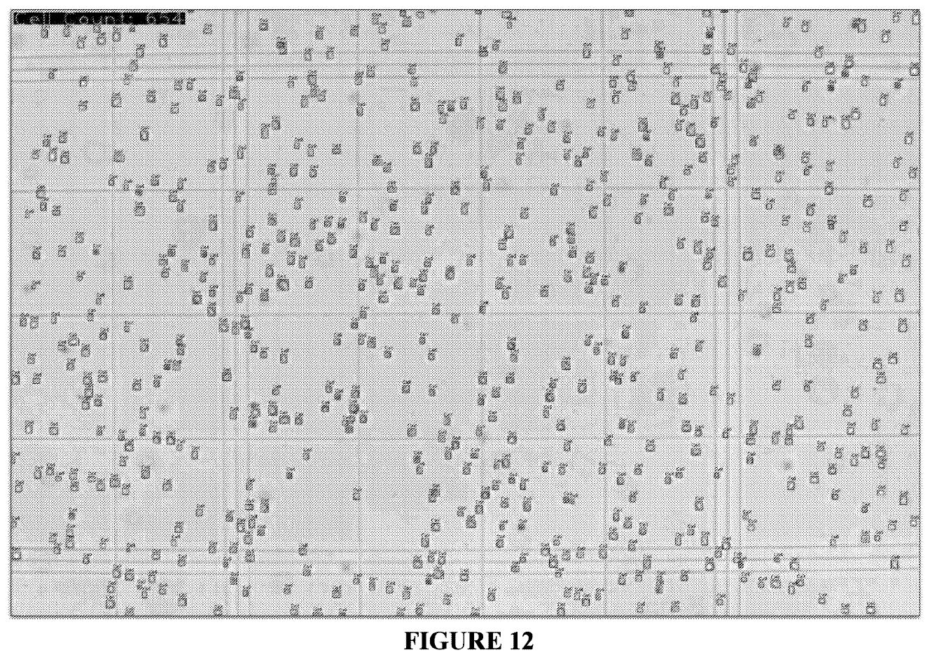

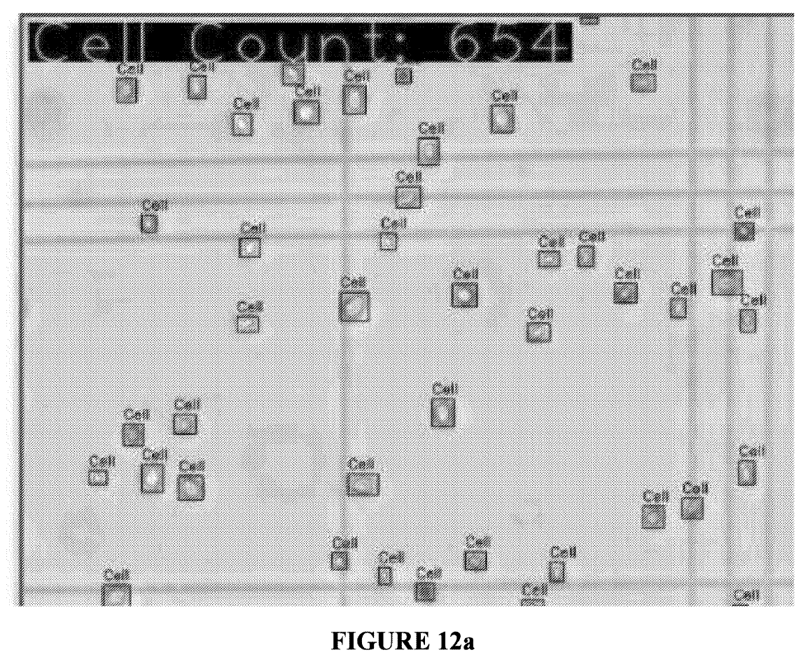

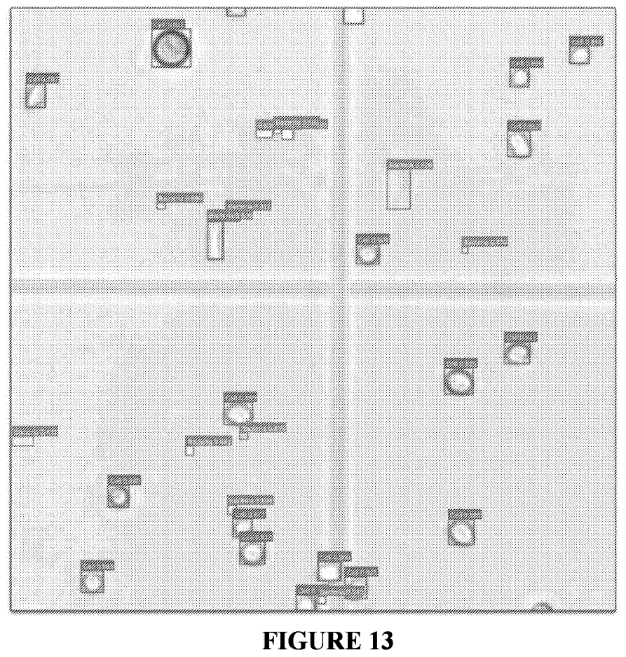

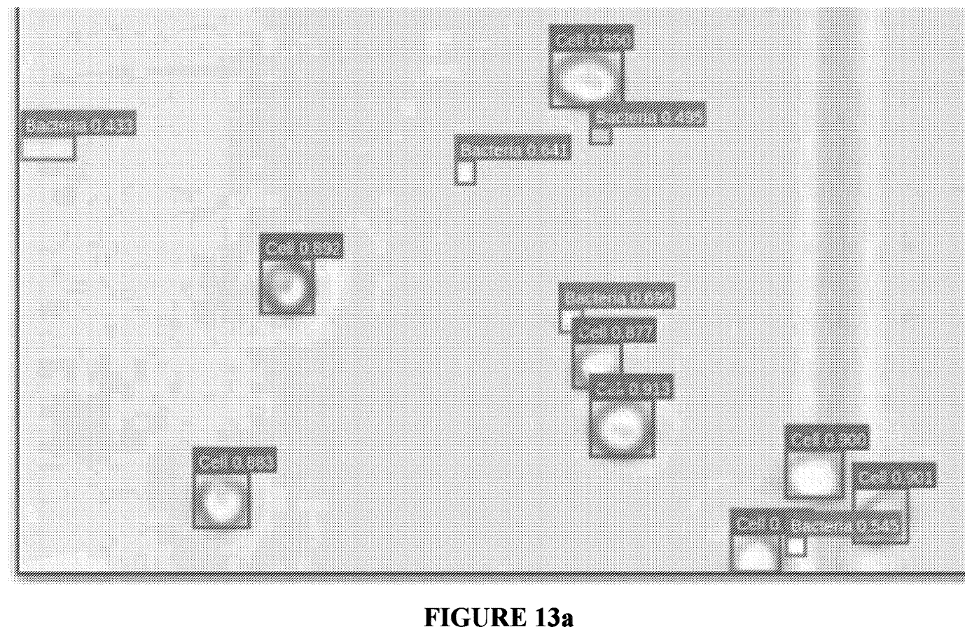

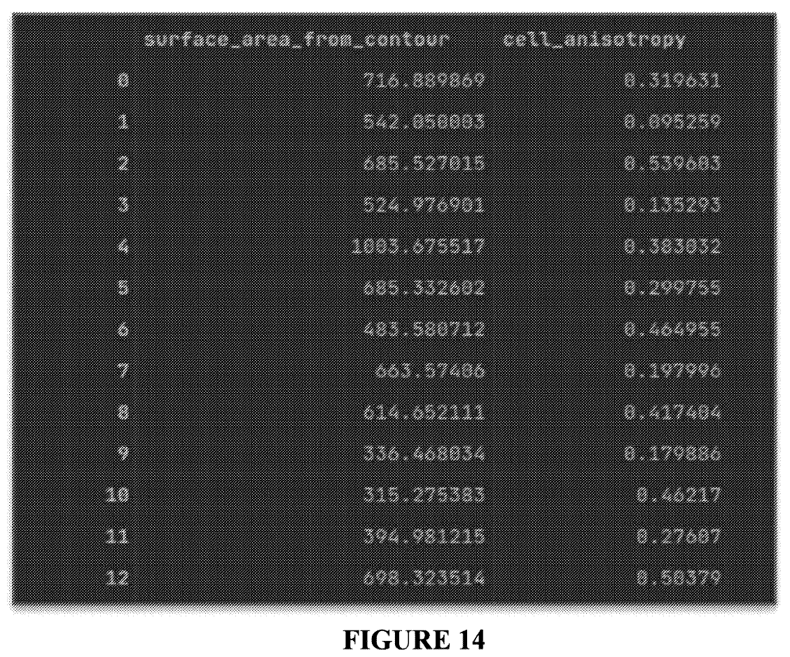

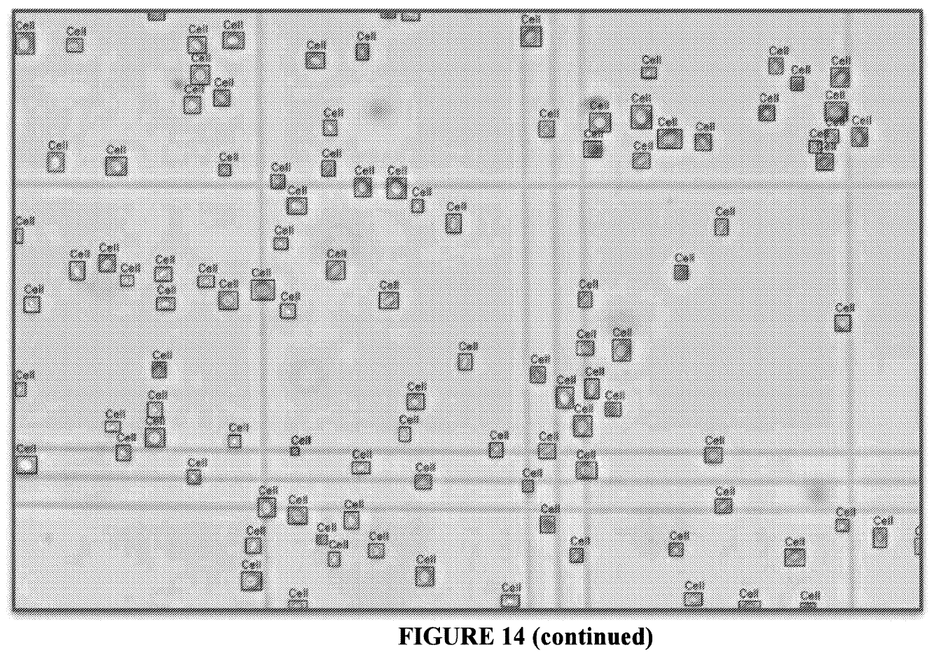

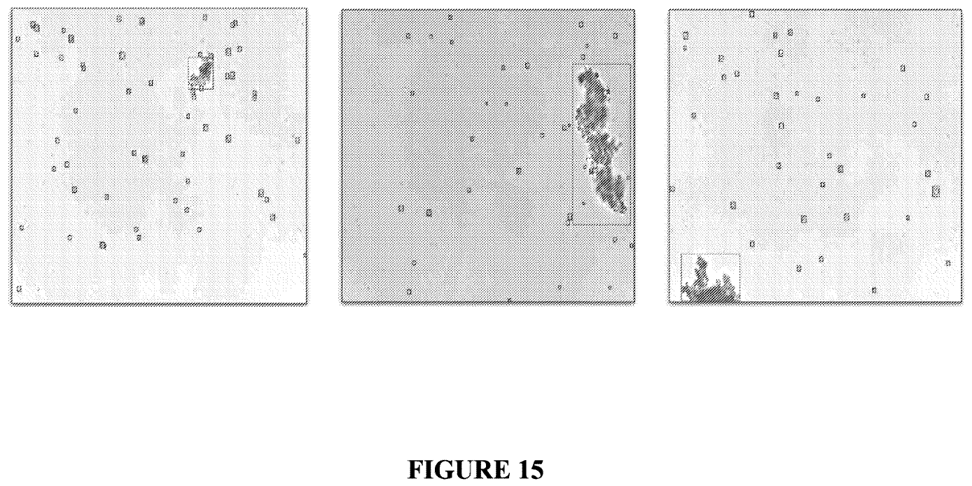

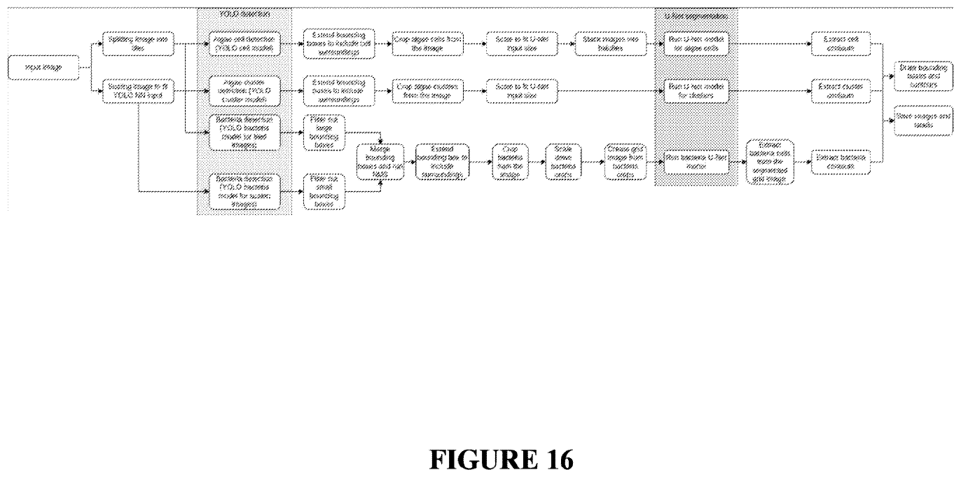

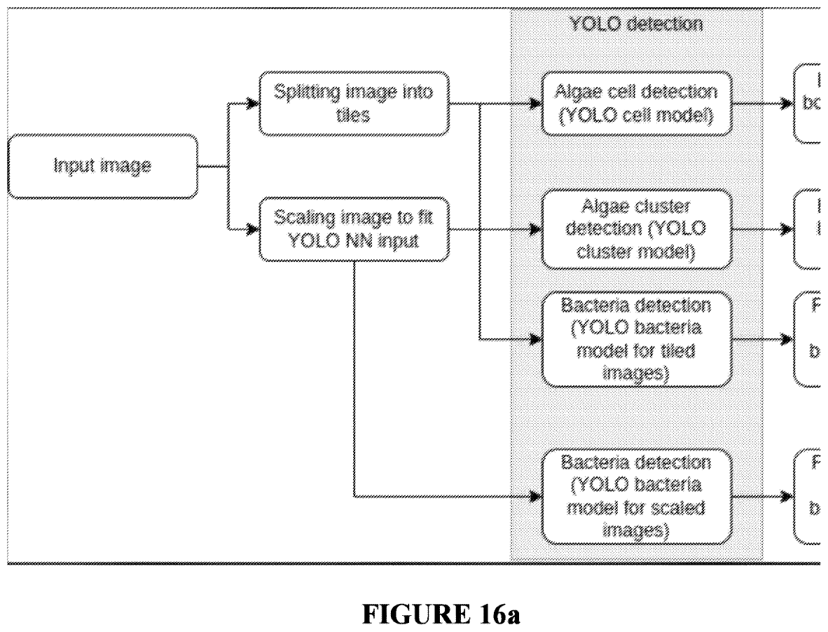

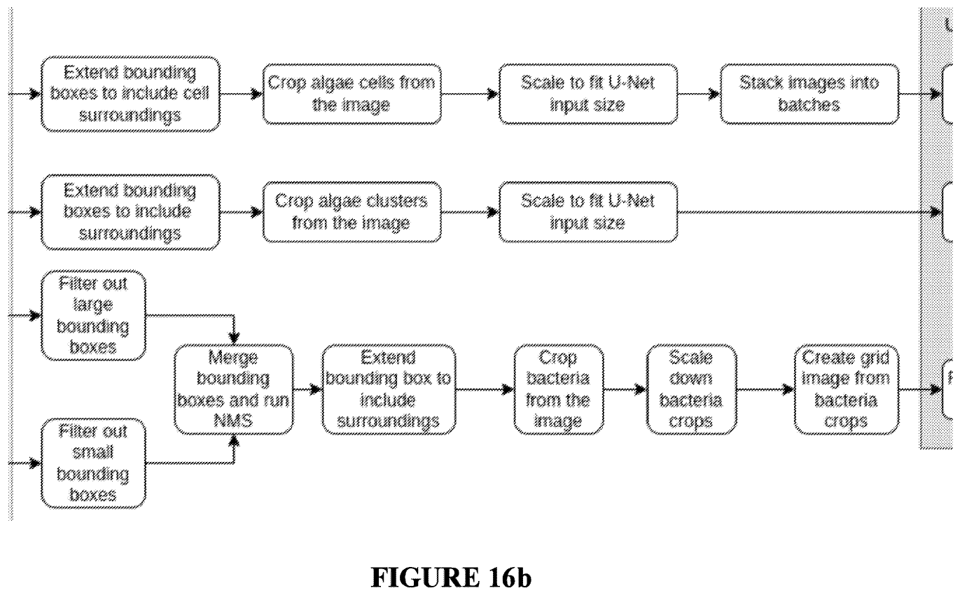

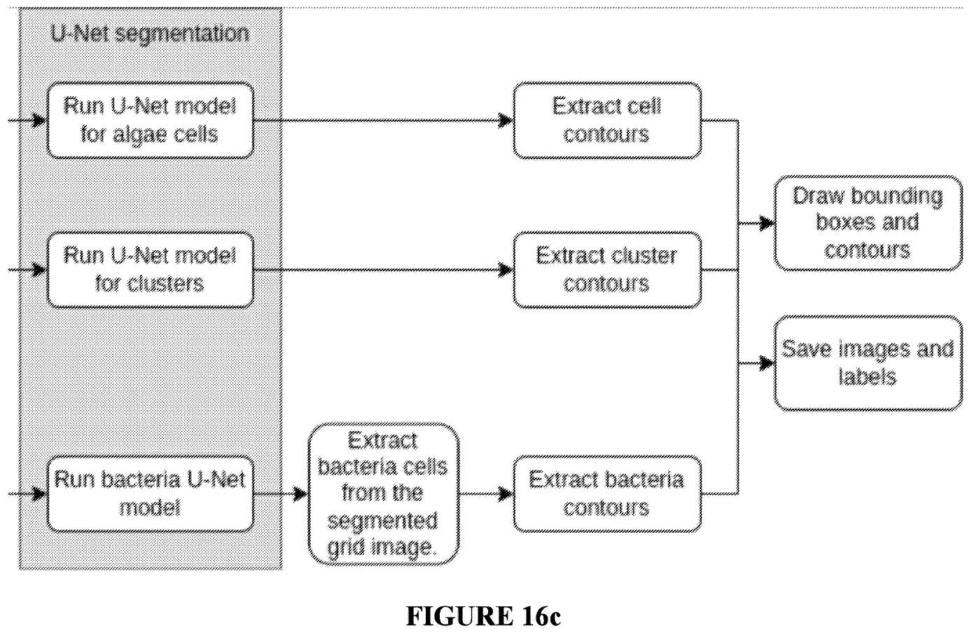

The image recognition system 6 carries out a first microalgae-cell detection step based on its ability to detect microalgae cell contour (see FIG. 11). The image recognition system 6 then carries out a subsequent microalgae-cell property recognition step. The microalgae-cell property recognition step can comprise: a counting step, to count the number of microalgae cells present in the image (see FIGS. 12 and 12a); or, a cell-feature recognition step whereby: algal cells and smaller bacterial cells are recognised (see FIGS. 13 and 13a); cell surface area and/or cell volume is calculated (see FIG. 14); or, cell clumps are recognised (see FIG. 15). The image recognition system 6 can utilise artificial intelligence (AI)/machine learning to carry out the detection and property recognition steps, and depicted in the flowchart of FIGS. 16, 16a, 16b and 16c.

A user of the receiver 40i can request the property of the microalgae growth that needs to be determined.

The image recognition system 6 can cause repositioning of the microscope's lens 40e such that the lens 40e automatically scans the depth of the channel 31b and preferentially focuses on microalgal cells 1a, 1b within the channel 31b. The image recognition system 6 can cause the microscope lens 40e to focus on the largest number of microalgae cells within the channel 31b. The image recognition system 6 can cause the lens 40e to focus through different depths/planes of the channel 31b, until it locates the highest number of microalgae cells. These steps can be carried out using artificial intelligence (AI)/machine learning.

As seen in FIG. 3, the pre-sampling wetting system 2 wets the microfluidic device 31 prior to sampling and conveying of the sample. The pre-sampling wetting system 2 includes a container 20 adapted to contain a wetting agent 21, a wetting line 22 for conveying the wetting agent 21 from the wetting agent container 20 to the sampling line 32 and microfluidic device 31, and various electric valves and flow diverters/3-way valves 27, 37.

The wetting agent 21 is water or other polar solvent, such as ethyl alcohol.

The wetting line 22 includes an inlet 22a connected to an outlet 20a of the wetting agent container 20, and an outlet 22b connected to the sampling line 32 upstream of the microfluidic device 31. The wetting line 22 comprises lengths of platinum cured silicone tubing connected together, having an internal diameter of approximately 0.6 mm.

The wetting system 2 further includes pump 33, which is located in-line downstream of the microfluidic device 31, such that the pump 33 can draw wetting agent 21 from the wetting agent container 20 to the microfluidic device 31 via part of the sampling line 32 to the microfluidic device 31.

The pre-sampling wetting system includes pump line 34.

The pre-sampling wetting system includes waste container 35. The waste container 35 can collect part or all of the wetting agent 21, if necessary.

The various electric valves and flow diverters/3-way valves 27, 37 control the flow of wetting agent 21 between the wetting agent container 20 and the microfluidic device 31, as well as from the microfluidic device 31 to the waste container 35.

As seen in FIG. 6-8, the sample-cleaning system 5 can remove the sample or most of the sample from the microfluidic device 31 and from the sampling line 32 after the imaging step. The sample-cleaning system 5 can both flush the microfluidic device 31 and the sampling line 32 with air, and further flush the microfluidic device 31 and at least part of the sampling line 32 with a cleaning agent 51.

The sample-cleaning system 5 includes a container 50 adapted to contain a cleaning agent 51, a cleaning line 52 for conveying the cleaning agent 51 from the cleaning agent container 50 to the sampling line 52 and microfluidic device 31, and various electric valves and flow diverters/3-way valves 27, 37.

The cleaning line 52 includes an inlet 52a connected to an outlet 50a of the cleaning agent container 50, and an outlet 52b connected to the sampling line 32 upstream of the microfluidic device 31. The cleaning line 52 comprises lengths of platinum cured silicone tubing connected together, having an internal diameter of approximately 0.6 mm.

The cleaning agent 51 is water or other polar solvent, such as ethyl alcohol.

The cleaning system 5 further includes pump 33, which is located in-line downstream of the microfluidic device 31, such that the pump 33 can draw cleaning agent 51 from the cleaning agent container 50 to the microfluidic device 31 via part of the sampling line 32 to the microfluidic device 31.

The cleaning system 5 includes pump line 34.

The cleaning system 5 includes waste container 35. The waste container 35 can collect part or all of the cleaning agent 51, if necessary.

The various electric valves and flow diverters/3-way valves 27, 37 control the flow of cleaning agent 51 between the cleaning agent container 50 and the microfluidic device 31, as well as between the microfluidic device 31 and the waste container 35.

To flush the microfluidic device 31 and the sampling line 32 with air, the operation of pump 33 can be reversed, in which case it can displace the sample from the microfluidic device 31 and sampling line 32 using air, and return the sample or most of the sample to the microalgae culture container 30.

As seen in FIG. 1, the controller 7 includes a controller housing 70 and logic circuitry 71. The controller is electrically connected to the pump 33, valves/flow diverters 27, 37, stepper motor 40h, imaging system's/imaging device's 4 electricals/electronics, and image recognition system 6. The controller 7 can utilise artificial intelligence (AI)/machine learning to carry out one or more functions/steps for automation.

In order to carry out an automated method of determining a property of microalgae growth, the following sequence of steps is carried out:
1. A microalgae culture 1 is grown within the container 30, as seen in FIG. 2.
2. A pre-sampling wetting step is carried out as seen in FIG. 3, whereby the pre-sampling wetting system 2 wets the microfluidic device 31 prior to sampling and conveying of the sample. The flow diverters/3-way valves 27, 37 are opened and the pump 33 is activated such that wetting agent 21 is drawn from the wetting agent container 20 through the wetting line 22, through part of the sampling line 32 and finally to the microfluidic device 31.
3. A sampling and conveying step is carried out as seen in FIG. 4, whereby a sample of the microalgae culture 1 from the container 30 is withdrawn and conveyed to the microfluidic device 31. The flow diverter/3-way valve 27 is opened and the pump 33 is activated such that culture 1 is drawn from the container 30 through the sampling line 32 and further to the microfluidic device 31. The sample or part thereof can also be conveyed to the waste container 35 via the pump line 34 and waste line 36. The sampling system 3 conveys the sample to the microfluidic device 31 such that microalgal cells 1 of the sample are present within the channel 31b for image capture. The pump 33 is used to draw in the sample and then stopped. As the flow of sample ceases, one or more images of microalgae cells of the sample can then be captured by the imaging device 4.
4. An imaging step is carried out as seen in FIGS. 1 and 5, whereby the imaging device 4 captures one or more images of microalgal cells 1 of the sample located within the channel 31b of the microfluidic device 31. Captured imaged data is then transmitted wirelessly to a receiver 40i, such as a computer or computer network (internet).
5. An image recognition step is carried out as seen in FIG. 1 using image recognition software executed on a receiver 40i. The image recognition system 6 processes captured image data of the microalgal cells 1 to determine a property of the microalgae growth. The image recognition system
6 can cause automatic repositioning of the microscope's lens 40e such that the lens 40e automatically scans the depth/different planes of the channel 31b and preferentially focuses on the largest number of microalgae cells within the channel 31b. The recognition step can include the step of outputting results to an Excel spreadsheet.

### ADVANTAGES

Advantages of one or more of the preferred embodiments include:

The automated cell analyser and method of the preferred embodiments can be used to extract and count/characterise microalgae on the micro level with no interaction from a human.

The automated cell analyser and method of the preferred embodiments can count and characterise microalgae more accurately and quicker than a human.

The automated cell analyser and method of the preferred embodiments can be taught to count and characterise microalgae cells of different strains.

The automated cell analyser and method of the preferred embodiments can be adapted for varying sizes and densities of microalgae.

The automated cell analyser and method of the preferred embodiments can discriminate microalgae from other small particles, such as debris, small stains and microbubbles.

The automated cell analyser and method of the preferred embodiments can be used with any suitable prokaryotic or eukaryotic cell, providing that it is unicellular or substantially unicellular which located within the microfluidic device.

### VARIATIONS

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

The terms “about” and “approximately” denote an interval of accuracy that a person skilled in the art will understand to still ensure the technical effect of the feature in question. The term typically indicates a deviation from the indicated numerical value of ±10%, preferably ±5%, more preferably ±2%, and even more preferably ±1%.

1. An automated cell analyzer for determining a property of a cell, comprising:
a sampling system capable of sampling a cell culture, comprising a microfluidic device, a sampling line for conveying a cell culture sample from a container adapted to contain the cell culture to a channel of the microfluidic device, and at least one pump located downstream of and in-line with the microfluidic device that moves the sample from the cell culture container to the channel, wherein the sample comprises at least one cell;
an imaging system associated with the microfluidic device, adapted to capture one or more images of the at least one cell of the sample located within the channel;
an image recognition system capable of a recognizing the at least one cell within the one or more captured images of the sample, to thereby determine a property of the at least one cell, wherein the image recognition system utilizes artificial intelligence/machine learning to determine the property of the at least one cell, and wherein the image recognition system carries out a first cell detection step based on the detection of cell contour, and a subsequent cell property recognition step;
a sample-cleaning system capable of cleaning the channel and the sampling line or part thereof, comprising at least one pump located downstream of and in-line with the microfluidic device that is operable to displace the sample from the microfluidic device and return the sample or most of the sample to the cell culture container, and is operable to convey a cleaning agent of the sample-cleaning system to the channel and the sampling line or part thereof; and
a controller capable of automating the sampling, imaging, image recognition and sample-cleaning systems.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. An automated cell analyzer for determining a property of a cell, comprising:
a sampling system comprising a microfluidic device comprising at least one channel adapted to receive a sample comprising at least one cell;
an imaging system associated with the microfluidic device, adapted to capture one or more images of the at least one cell of the sample located within the at least one channel of the microfluidic device;
an image recognition system capable of recognizing the at least one cell within the one or more captured images of the sample, to thereby determine a property of the at least one cell; and
a controller capable of automating at least the imaging and image recognition systems.

21. An automated method of determining a cell property, comprising:
at least one sampling step, comprising conveying a sample containing at least one cell to a microfluidic device associated with an imaging system;
at least one imaging step, comprising using the imaging system to capture one or more images of the at least one cell of the sample located within the microfluidic device; and
at least one image recognition step, comprising recognizing the at least one cell within the one or more captured images of the sample, to thereby determine a property of the cell,
wherein at least the imaging and image recognition steps are automated.

22. (canceled)

23. (canceled)

24. (canceled)

25. The analyzer of claim 1, wherein:
the property of the cell comprises one or more properties selected from the group consisting of: the degree of cell growth; cell count; cell health; cell morphology; the level of recombinant protein expression by the cell; the level of biomolecule expressed by the cell; cell maturity; and, one or more traits of the cell;
the cell property recognition step is a step selected from the group consisting of: a counting step, to count the number of cells present in the one or more captured images; a cell-feature recognition step; and combinations thereof,
wherein the cell-feature recognition step is selected from the group consisting of: (a) recognizing parent and smaller daughter cells in close proximity of each other; (b) calculating cell surface area and/or cell volume; (c) recognizing cell clumps; (d) recognizing cell color; (e) discriminating between two different cell types; (f) identifying the cell species or strain type; and combinations thereof, and
the at least one cell comprises one or more cells selected from the group consisting of: a unicellular organism; a prokaryotic cell; a eukaryotic cell; a separate cultured cell of a multi-cellular organism; a microalgal cell; and combinations thereof.

26. The analyzer of claim 20, wherein the imaging system comprises an imaging device comprising:
optics in close proximity of the at least one channel of the microfluidic device such that the at least one cell of the sample within the at least one channel is imaged; and
a light source capable of illuminating the at least one cell of the sample within the at least one channel.

27. The analyzer of claim 26, wherein the optics comprise a lens and a lens positioning mechanism capable of infinite positioning of the lens relative to the at least one channel, and wherein the positioning and focusing of the lens is carried out automatically.

28. The analyzer of claim 27, wherein the imaging device comprises an inverted light microscope whereby the light source of the inverted light microscope is located above the at least one channel and the optics of the inverted light microscope are located beneath the at least one channel.

29. The analyzer of claim 20, wherein the imaging system comprises a digital camera capable of capturing image data in the form of a still image and/or video recording of the at least one cell of the sample.

30. The analyzer of claim 29, wherein the image recognition system comprises image recognition software that, when executed, processes captured image data of the at least one cell to determine the property of the at least one cell.

31. The analyzer of claim 30, wherein the image recognition system utilizes artificial intelligence-machine learning to determine the property of the at least one cell.

32. The analyzer of claim 31, wherein the image recognition system carries out a first cell detection step based on a detection of cell contour of the at least one cell.

33. The analyzer of claim 32, wherein the image recognition system carries out a cell property recognition step subsequent to the first cell detection step.

34. The analyzer of claim 33, wherein the cell property recognition step comprises a counting step, to count the number of cells present in the image.

35. The analyzer of claim 33, wherein the cell property recognition step comprises a cell-feature recognition step selected from the group consisting of: (a) whereby parent and smaller daughter cells in close proximity of each other are recognized; (b) whereby cell surface area and/or cell volume is calculated; (c) whereby cell clumps are recognized; (d) whereby cell color is recognized; (e) whereby two different cell types are discriminated between; (f) whereby a cell species or strain type is identified; and combinations thereof.

36. The analyzer of claim 27, wherein the image recognition system controls positioning and focusing of the lens such that the lens automatically scans a depth of the at least one channel and preferentially focuses on any cells within the at least one channel.

37. The analyzer of claim 36, wherein focusing and scanning of the lens is carried out automatically using artificial intelligence-machine learning.

38. The analyzer of claim 20, wherein the at least one cell comprises one or more cells selected from the group consisting of: a unicellular organism; a prokaryotic cell; a eukaryotic cell;
a separate cultured cell of a multi-cellular organism; a microalgal cell; and combinations thereof.

39. The analyzer of claim 20, wherein the property comprises one or more properties selected from the group consisting of: the degree of cell growth; cell count; cell health; cell morphology; cell species identification; cell strain identification; a level of recombinant protein expression by the cell; a level of a biomolecule expressed by the cell; cell maturity; and, one or more traits of the cell.

40. The method of claim 21, wherein the method is carried out using the automated cell analyzer of claim 1.

41. The method of claim 21, wherein the method is used for periodically sampling a microalgae cell culture, so as to automatically optimize growth of microalgae within the cell culture.
